(19) United States
(12) Patent Application Publication
(10) Pub. No.: US 2015/0204836 A1
(43) Pub. Date: Jul. 23, 2015

(54) SOLUBLE METAL SENSOR COMPOUNDS AND METHODS FOR MAKING AND USING THE SAME

(71) Applicants: Glenn D. PRESTWICH, (US); Ling ZANG, Salt Lake City, UT (US); Liu Xiaohui, Holladay, UT (US); UNIVERSITY OF UTAH RESEARHC FOUNDATION, Salt Lake City, UT (US)

(72) Inventors: Glenn D Prestwich, Eastsound, WA (US); Ling Zang, Salt Lake City, UT (US); Xiaohui Liu, Holladay, UT (US)

(73) Assignee: UNIVERSITY OF UTAH RESEARCH FOUNDATION, Salt Lake City, UT (US)

(21) Appl. No.: 14/420,910

(22) PCT Filed: Aug. 30, 2013
(86) PCT No.: PCT/US13/57448

§ 371 Date: Feb. 11, 2015

Related U.S. Application Data
(60) Provisional application No. 61/695,365, filed on Aug. 31, 2012.

(51) Int. Cl. G01N 33/18; C07D 487/04

(52) U.S. Cl. CPC G01N 33/1813; C07D 487/04

(57) ABSTRACT

Described herein are the preparation and use of metal sensor compounds in detecting metals that are toxic to humans or to the environment. In one aspect, the metal sensor compounds comprise a polycyclic aryl group (PAC), wherein at least one solubilizing group and at least one metal binding ligand are covalently bonded to (PAC).

### CROSS REFERENCE TO RELATED APPLICATIONS

This application claims priority upon U.S. provisional application Ser. No. 61/695,365, filed Aug. 31, 2012. This application is hereby incorporated by reference in its entirety for all of its teachings.

### ACKNOWLEDGEMENTS

The research leading to this invention was funded in part by the National Science Foundation, Grant Nos. CHE 0931466 and 1113373. The U.S. Government has certain rights in this invention. The U.S. Government has certain rights in this invention.

### BACKGROUND

Over the past thousands of years man has learned to use metals for various purposes. Advances in metallurgy and the use of metals in industry have resulted in significant technological leaps for mankind. Historians often refer to the Stone Age, the Bronze Age and the Iron Age because of the defining importance of these substances in societies during these time periods. Metals such as gold, silver and platinum have been used as a standard currency or as a common denominator for currency throughout history. Other metals have been employed for the benefit of mankind through numerous technological uses. This has resulted in widespread mining for metal containing ores and their distribution throughout the world. Slowly, it was realized that metals can be harmful to the health of humans and the constituents of the environment in which they reside. A group of metals referred to as heavy metals are of particular concern because of the serious health problems they can cause to humans and their widespread distribution in the environment due to their use. Heavy metals are often released into the environment because of the mining of the ores, petroleum processing and refining, coal burning, cement production and other industry manufacturing. Serious heavy metal culprits include lead (paint, petroleum additive, water delivery, and food), cadmium (tobacco smoking, phosphate fertilizers, sewage sludge, nickel cadmium (NiCd) batteries, plating, pigment, and plastics), arsenic (wood preservative, pressure treated lumber, pesticide, presence in drinking water due to widespread distribution in earth's crust) and mercury (discussed in more detail below) due to their use in human industry and their broad presence in the environment.

Metal contamination can be particularly problematic when it is in proximity to water since this can greatly increase the chances and extent of exposure to humans. Toxic metals in water sources are becoming increasingly relevant as the world's population grows further taxing water supplies. As more and more metal contaminants end up in this precious resource, which is the basis for life on earth, there is a greater need for methods to detect these poisons in a rapid, easy-to-use and portable format due to their widespread distribution. An example of a metal contaminant that is particularly dangerous is mercury, which has the chemical symbol “Hg” that is derived from the word hydrargyrum (“hydr” meaning water and “argyros” meaning silver). Mercury is present in various forms in the environment including organic forms (e.g., methylmercury) and inorganic forms (e.g., elemental Hg(0), Hg⁺ (e.g., Hg₂Cl₂), Hg²⁺ (e.g., HgO, HgCl₂, HgS)), many of which are toxic.

Hg²⁺ (also designated Hg⁺⁺, Hg(II) or mercuric mercury) is a highly toxic metal ion that causes serious health and environmental problems. Hg²⁺ is toxic and is readily converted to other forms of mercury which are also toxic. Increasingly, governments are recognizing the need to monitor and regulate the amount of Hg²⁺ released into the environment. Acceptable concentrations of Hg²⁺ are quite low since it is highly toxic to humans. For example, in order to provide safe drinking water, inorganic mercury levels should be less than 2 ppb (or 10 nM). In analogy to cancer, early diagnosis at low level of poisoning leads to more successful treatment.

To detect such trace amounts of Hg²⁺ with minimal false positives, a sensor technique with extremely high selectivity is needed. Achieving selectivity can be very difficult because Hg²⁺ ions co-occur in nature with other physiologically important metal ions such as Cu²⁺, Ni²⁺, Fe²⁺, Fe³⁺, Zn²⁺, Mn²⁺, Mg²⁺, Ca²⁺, Ba²⁺, K⁺, and Na²⁺. Moreover, contaminated water samples may include Cd²⁺, Pb²⁺, Ba²⁺, Ni²⁺, Cr²⁺, Co²⁺, an ion of arsenic, antimony, or thallium, a rare earth metal ion, and a lanthanide ion, the presence of which may also be detrimental to human health. It is relevant to differentiate amongst metal ions such as these and detect Hg²⁺ ion selectively. In addition, the concentration of these other ions may be much higher than that of Hg²⁺, which presents a significant challenge to accurately detecting and measuring Hg²⁺ concentration. Sources of mercury contamination include, but are not limited to, burning of fossil fuels, petroleum and natural gas processing, manufacturing processes using mercury reagents, production of cement, amalgams used in dentistry, and amalgamation processes associated with mining. Mercury contamination can accumulate in the environment through the processes of food chain or bioaccumulation, in which alkyl mercury compounds can be formed either through bioreduction of mercuric ions (Hg²⁺) or biooxidation of metallic mercury (Hg). Alkyl mercury compounds persist in biological tissues. Consumption of alkyl mercury containing prey organisms by predators leads to accumulation of increasing tissue mercury levels in higher predators, particularly birds and fish.

In view of the wide variety of sources of mercury contamination and the widespread geographic distribution of mercury contamination, there is a pressing need for rapid, user friendly, highly selective and highly sensitive detection systems for this very toxic metal.

### SUMMARY

Described herein are the preparation and use of metal sensor compounds in detecting metals that are toxic to humans or to the environment. In one aspect, the metal sensor compounds comprise a polycyclic aryl group (PAC), wherein at least one solubilizing group and at least one metal binding ligand are covalently bonded to (PAC).

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The following description can be better understood in light of the Figures.

FIG. 1 shows fluorescence response of Compound 1 with Hg²⁺ or 11 other metal ions. See Example 2.

FIG. 2 shows a linear detection response for Hg²⁺ in the indicated ranges. See Example 3.

FIG. 3 shows a Job plot for determining the stoichiometry of binding of Compound 1 to Hg²⁺. See Example 6.

FIG. 4 shows the fluorescence response of Compound 1 with a mixture of 11 other metal ions and Hg²⁺ as a function of pH. See Example 5 for definition of lines A, B, and C.

FIG. 5 shows a stability test of Compound 1. See Example 4.

FIG. 6 shows an exemplary scheme (Route/Scheme A) for the synthesis of compounds as described herein. See Example 1.

FIG. 7 shows an exemplary scheme (Route/Scheme B) for the synthesis of compounds as described herein. See Example 1.

FIG. 8 shows an exemplary scheme (Route/Scheme C) for the synthesis of compounds as described herein. See Example 1.

FIG. 9 shows Compounds 3, 4, 5, and 6. See Example 1 and FIG. 10.

FIG. 10 shows exemplary schemes for the synthesis of Compounds 3, 4, 5, and 6, as described herein. See Example 1.

FIG. 11 shows a synthetic procedure for making metal sensor compounds having different groups at the 1H-pyrimidine-2,4-dione ring.

FIG. 12 shows the weight of droplets from LDPE squeeze bottles containing various concentrations of glycerol-water (15%, 30%, 50%).

FIG. 13 shows fluorescence intensity of 1.0 μM Compound 1 (black) measured at 596 nm in the presence of 2.0 μM Hg²⁺ (gray) at varying temperatures.

### DETAILED DESCRIPTION

The following description supplies specific details in order to provide a thorough understanding of the compounds and methods described herein. Nevertheless, the skilled artisan would understand that the compounds (and particularly the tunable metal sensor compounds) and associated methods of making and using such compounds can be implemented and used without necessarily employing these specific details. Indeed, the metal sensor compounds and associated methods can be placed into practice by modifying the illustrated compounds and methods and can be used in conjunction with any other materials and techniques conventionally used in the industry. For example, while the description refers to metal sensor compounds and in particular aspects mercury sensing, it could be modified to be used with other metals or analytes. Additionally, the metal sensor compounds may be used in applications related to electronics, dyes, pigmentation, photovoltaic applications, nanotechnology, mining operations, environmental monitoring and clean-up etc.

The metal sensor compounds herein can be used for a number of applications. The metal sensor compounds described herein are composed of a number of covalently linked moieties chosen so that in combination they: (1) do not interfere substantially with the relevant spectral properties (e.g., absorbance, fluorescence and quenching properties) of the metal sensor compound used for detection purposes; (2) do not substantially interfere with the binding of the metal sensor compound to the metal ion (e.g., selectivity and sensitivity); (3) give the molecule a desired solubility profile; and (4) give the molecule acceptable or desirable stability in solution or as a solid.

The metal sensor compounds are tunable in terms of a number of properties including metal ion binding selectivity, metal ion binding sensitivity, solubility, fluorescence properties, stability, optimal pH, and any other property deemed relevant for a particular use. The tunable features of the metal sensor compounds as described herein may be achieved through selecting or defining a variety of parameters including, the specific chemical nature of the compound by varying the chemical moieties that the compound is comprised of, altering the concentration of the compound in a particular solvent (or solvent system), altering the solvent (or solvent system), altering the absorbance or emission or quenching properties of the compound, altering the pH of the solution, altering the ionic strength of solution, altering the temperature of the solution, etc. Thus, a platform of metal sensor compounds is provided herein as well as metal sensor systems.

### DEFINITIONS

Unless defined otherwise, all technical and scientific terms used herein have the same meaning as is commonly understood by one of ordinary skill in the art.

As used herein, the term “metal sensor solution” refers to a solution having a particular concentration of metal sensor compound.

As used herein, the term “calibration solution” refers to a solution having a particular concentration of a metal or metal ion that is meant to be detected by the metal sensor compound (the metal or metal ion generally in the concentration range of 0.01 μM to 100 μM). The calibration solution can comprise an organic solvent or solvent system, the calibration solution can comprise an aqueous solvent, or the calibration solution comprises an aqueous solvent that is a mixture of an organic solvent (or solvent systems) and water depending on the metal (or metal ion) and nature of the application for which it is being used.

As used herein, the term “test sample” refers to a sample that is intended to be tested (e.g., determination of presence and/or level or concentration) for having a metal or metal ion.

As used herein, the term “stock solution” refers to a more concentrated form of a metal sensor solution or calibration solution that is to be diluted prior to use in metal sensing assays. For example, the stock solution has a predetermined concentration of metal (e.g., metal cation) or metal sensor compound that is diluted 1000-fold, 100-fold, or 10-fold prior to use in the metal sensing assays for calibration or testing a test sample.

As used herein, the term “test solution” refers to a solution having (a) a test sample (“test sample solution” when combined with component (b)) or a calibration solution (“test calibration solution” when combined with component (b)) and (b) metal sensor solution. A “test solution container” is a container that is used in the detector e.g., a cuvette. The test solution can be at any pH depending on the nature of the metal or metal ion to be detected and the metal sensor compound. Desirably, the test solution is at a pH that achieves a selected metal or metal ion binding sensitivity and selectivity.

As used herein, the term “contacting” in specific reference to contacting a compound or solution with another compound or solution refers generally to combining or mixing.

As used herein, the term “alkyl” refers to a straight-chain or branched-chain alkyl group containing from 1 to 20 carbon atoms. A (C1-C10) alkyl has from 1 to 10 carbon atoms and a (C1-C6) alkyl has from 1 to 6 carbon atoms and a (C1-C4) alkyl has from 1 to 4 carbon atoms. Examples of alkyl groups include, but are not limited to, methyl, ethyl, n-propyl, isopropyl, n-butyl, isobutyl, sec-butyl, tert-butyl, pentyl, isopentyl, neo-pentyl, iso-amyl, hexyl, heptyl, octyl or nonyl.

As herein, the term “cycloalkyl” refers to an all-carbon monocyclic or fused ring (i.e., rings which share an adjacent pair of ring carbon atoms) group. Examples, without limitation, include cyclopropane, cyclobutane, cyclopentane, cyclohexane, and cycloheptane.

As used herein, the term “alkylenyl” or “alkylene” refers to an alkyl group attached at two positions, i.e. an alkanediyl group. Examples include, but are not limited to, methylene, ethylene, propylene, butylene, pentylene, hexylene, heptylene, octylene, or nonylene.

As used herein, “halogen” or “halo” refers to a chloro (or —Cl), bromo (—Br), fluoro (or —F) or iodo (—I) group.

As used herein, the term “amino acid” refers to a molecule that contains both amine and carboxyl functional groups and includes both natural and non-natural amino acids. Amino acids can be racemic or not optically active or are typically “D” or “L” optically active isomers. Amino acids can be alpha-amino acids, beta-amino acids, and gamma-amino acids. Alpha amino acids have the amino and carboxylate groups attached to the same carbon (called the α-carbon). The various naturally occurring alpha-amino acids differ in which side chain (R group) is attached to their alpha carbon which can vary in size from just a hydrogen atom in glycine, through a methyl group in alanine, to a large heterocyclic group in tryptophan. 20 naturally occurring alpha-amino acids include alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y)) all of which have the “L” stereochemical configuration about the alpha carbon except glycine which is not optically active. Other amino acids (not necessarily alpha-amino acids) include, but are not limited to, hydroxyproline, γ-carboxyglutamate, O-phosphoserine, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine, 2,4-diaminoisobutyric acid, desmosine, 2,2′-diaminopimelic acid, 2,3-diaminoproprionic acid, N-ethylglycine, N-methylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, pentylglycine, pipecolic acid, thioproline, methionine sulfoxide, methionine sulfone, S-(carboxymethyl)-cysteine, 5-(carboxymethyl)-cysteine sulfoxide, S-(carboxymethyl)-cysteine sulfone, aspartic acid-(beta-methyl ester), N-ethylglycine, alanine carboxamide, homoserine, norleucine, and methionine methyl sulfonium. Beta-amino acids have an extra ethylene group in between the amino group and the carboxyl croup whereas gamma-amino acids have a propylene group in between the carboxyl and amino functionalities. The intervening ethylene or propylene groups may be substituted or unsubstituted. Preferred substitutions for the beta- and gamma-amino acids are the R-groups described in the list of specific amino acids listed above.

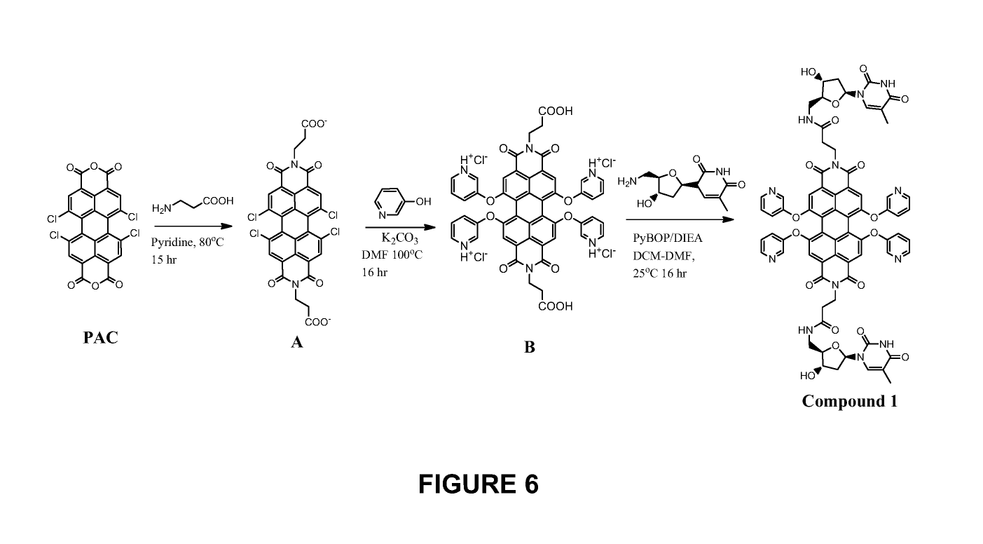

As used herein, the term “amino acid moiety derived” refers to the resulting moiety that is derived from the use of the amino acid in a particular synthetic context. For example, “amino acid moiety derived” from beta-alanine refers to —CH₂—CH₂—C(═O)— when the amino acid beta-alanine (—NH₂—CH₂—CH₂—C(═O)OH) is condensed or reacted with perylene (see e.g., FIG. 6).

As used herein, “heterocyclyl” or “heterocycle” each refer to a saturated, partially unsaturated, or fully unsaturated monocyclic, bicyclic, or tricyclic group containing at least one heteroatom as a ring member, wherein each said heteroatom may be independently selected from the group consisting of nitrogen, oxygen, and sulfur wherein the nitrogen or sulfur atoms may be oxidized (e.g., —N(═O)—, —S(═O)—, —S(═O)₂—). Additionally, 1, 2 or 3 of the carbon atoms of the heterocyclyl may be optionally oxidized (e.g., to give an oxo group or —C(═O)—). Heterocyclyls typically (but not always) do not have more heteroatoms as compared to carbon atoms as ring constituents. Heterocyclyls can have from 1 to 4 heteroatoms as ring members is some aspects. Heterocyclyls can have from 1 or 2 heteroatoms as ring members is some aspects. Heterocyclyls typically have from 3 to 8 ring members in each ring which can be shared with other ring systems in the case of bicyclic or tricyclic heterocyclyls (which may or may not be fused (e.g., where two rings share at least 2 ring atoms)). Yet another group of heterocyclyls has from 3 to 7 ring members in each ring. Again another group of heterocyclyls has from 5 to 6 ring members in each ring. “Heterocyclyl” is intended to encompass a heterocyclyl group fused to benzo ring system or other ring systems having only carbon as ring members. Examples of heterocyclyl groups include, but are not limited to, pyrrolidinyl, tetrahydrofuranyl, dihydrofuranyl, tetrahydrothienyl. tetrahydropyranyl, dihydropyranyl, tetrahydrothiopyranyl, piperidino, morpholino, thiomorpholino, thionyl, piperazinyl, homopiperazinyl. azetidinyl. oxetanyl, thietanyl, homopiperidinyl, oxepanyl, thiepanyl, oxazepinyl, diazepinyl, thiazepinyl, 2-pyrrolinyl, 3-pyrrolinyl, indolinyl, pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, or imidazolidinyl. Examples of “heteroaryls” that are heterocyclyls include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl. quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, or furopyridinyl.

As used herein, the term “optionally substituted” or “optional substituents” refers to no additional substituents on the indicated moiety or molecule or any substituent or substituents that do not undesirably or unacceptably interfere with a selected property or characteristic of the compound. In a more specific definition, optionally substituted or optional substituents refer to 0, 1, 2, or 3 independently selected substituents. Although other optional substituents may be used, one set of optional substituents is hydroxyl, halo, (═O), —S(═O)₂((C1-C3)alkyl), —CHF₂, —OCF₃, —OCHF₂, —CF₃, —SO₃H, and oligoethylene oxide.

As used herein, the term “oligoethylene oxide” refers to a polymer have from 2 to 20, 2 to 15, 2 to 10 or 2 to 5 repeating units of the formula (—CH₂—CH₂—O—). The terminal ethylene group may be a free hydroxyl group, —OH, or may be capped with an alkyl group. In some aspects, the alkyl group is a methyl or ethyl group

As used herein, the term “substituted” refers to at least one substituent on the indicated moiety or molecule that does not undesirably or unacceptably interfere with a selected property or characteristic of the compound. In some cases, but not all cases, 1, 2, or 3 substituents are present and are selected from hydroxyl, halo, (═O), —S(═O)₂((C1-C3)alkyl), —CHF₂, —OCF₃, —OCHF₂, —CF₃, —SO₃H, and oligoethylene oxide.

As used herein, “sugar” means a monosaccharide, disaccharide, oligosaccharide, or polysaccharide. Glycosaminoglycans are a particular type of polysaccharide that includes amino substituents on the sugar.

As used herein, a “water miscible organic non-volatile polymer” refers to a polymer in which the metal sensor compound is incorporated (e.g., dispersed, interspersed, mixed, or combined) that when exposed to test sample containing a metal ion of interest (e.g., liquid or fluid) is capable of quenching the fluorescence produced by the metal sensor compound depending on the concentration of selected metal ion in the test sample and the amount of metal sensor compound incorporated into the water miscible organic non-volatile polymer.

Compounds

Described herein are metal sensor compounds comprising a polycyclic aryl group (PAC), wherein at least one solubilizing group and at least one metal binding ligand are covalently bonded to (PAC). The compounds are useful for detecting metals or metal ions in solution including aqueous solutions. In one specific implementation, a metal sensor compound that is soluble in water (or a water-miscible organic solvent) and is capable of fluorescing in aqueous solution is provided. When the compound is exposed to, or contacted with, a metal ion it forms a complex with the metal ion which results in a detectable change in a spectral property of the compound (e.g., alteration or quenching of fluorescence).

In one aspect, the metal sensor compounds described herein have the Formula (I):

(X-L₁)ₙ-(PAC)-(L₂-Z)ₘ (I)

or a salt or solvate thereof, wherein
L₁ is a linker that covalently bonds each X to (PAC);
L₂ is a linker that covalently bonds each Z to (PAC);
X is a solubilizing moiety;
n is from 1-10;
Z is a metal binding ligand;
m is from 1-10; and
(PAC) is a polycyclic aryl group.
Each group in Formula (I) is described in detail below.

As used herein, “polycyclic aromatic core” or “(PAC)” refers to a compound having two or more fused ring systems that are completely aromatic or partially aromatic. In some aspects, the polycyclic aryl group (PAC) is a group that in combination with the remaining moieties on the metal sensor compound, exhibits desirable spectral properties (e.g., absorbance, fluorescence and fluorescence quenching properties). Furthermore, the (PAC) group is selected such that the metal sensor compound is stable in solution and as a solid when in an appropriate container. In addition to the linkers, solubilizing moieties, and metal binding ligands described herein, the polycyclic aryl group can be optionally substituted with one or more groups. Non-limiting examples of such substituents include, but are not limited to, halogens (e.g., —Cl, —Br, —F and —I), oxo (e.g, ═O), —OH, and oligoethylene oxide. It is contemplated that other hydrophilic polymers may be used in a similar manner to oligoethylene oxide. Furthermore, the rings atoms of the polycyclic aryl group can be but need not all be carbons, e.g., one or more of the ring atoms can be a heteroatom selected from oxygen, nitrogen or sulfur. According to some aspects, the (PAC) group and its substituents as defined here can be selected for optimizing properties for use in other applications (e.g., not metal sensing applications). Preferably, the (PAC) group and its substituents may be chosen to tune or optimize the spectral properties of the metal sensor compound.

In one specific aspect, the (PAC) group is a tetracarboxylic diimide. In one aspect, the (PAC) group is perylene or a perylene derivative or analog having 2 or more of the carbons of the fused polycyclic ring system replaced with a heteroatom chosen from oxygen, sulfur, or nitrogen. The (PAC) group, in some aspects, is a tetracarboxylic diimide. In some aspects, the tetracarboxylic diimide is perylene-3,4,9,10-tetracarboxylic diimide. In some aspects, the metal sensor compound is N-(((2R,3S,5R)-3-hydroxy-5-(5-methyl-2,4-dioxo-3,4-dihydropyrimidin-1(2H)-yl)tetrahydrofuran-2-yl)methyl)-3-(9-(3-(4(2S,3R,5S)-3-hydroxy-5-(5-methyl-2,4-dioxo-3,4-dihydropyrimidin-1(2H)-yl)tetrahydrofuran-2-yl)methyl)amino)-3-oxopropyl)-1,3,8,10-tetraoxo-5,6,12,13-tetrakis(pyridin-3-yloxy)-9,10-dihydroanthra[2,1,9-def:6,5,10-d′e′f′]diisoquinolin-2(1H,3H,8H)-yl)propanamide or 2-((2R,3S,5R)-3-hydroxy-5-(5-methyl-2,4-dioxo-3,4-dihydropyrimidin-1(2H)-yl)tetrahydrofuran-2-yl)methyl)-9-(((2S,3R,5S)-3-hydroxy-5-(5-methyl-2,4-dioxo-3,4-dihydropyrimidin-1(2H)-yl)tetrahydrofuran-2-yl)methyl)-5,6,12,13-tetrakis(pyridin-3-yloxy)anthra[2,1,9-def:6,5,10-d′e′f′]diisoquinoline-1,3,8,10(2H,9H)-tetraone.

The compounds described herein have linkers for covalently attaching the solubilizing moiety and metal binding ligand to (PAC). These are referred to as L₁ and L₂, respectively, in Formula I. The linker (i.e., L₁ and L₂) in combination with the remaining moieties of the compound of Formula (I), (1) does not unacceptably alter the spectral properties (e.g., absorbance, fluorescence and fluorescence quenching properties) of the molecule, (2) provides acceptable or desirable solubility characteristics, (3) provides for acceptable or desirable metal ion binding sensitivity, and (4) provides for acceptable or desirable metal ion binding selectivity.

Referring to Formula (I), L₁ covalently attaches each water solubilizing moiety X to (PAC). In one aspect, L₁ is alkylenyl, —O—, —S—, —NR₁₀— (where R is hydrogen or an alkyl group), and oligoethylene oxide. When the metal sensor compound has more than one L₁, e.g., n is greater than 1, these groups can be chosen independently, that is to say they can be different, or they can be the same.

The second linker L₂ in Formula (I) covalently attaches each metal binding ligand Z to (PAC). In one aspect, L₂ includes one or more amino acid moieties or amino acid derived moieties. In one aspect, L₂ is an alpha-amino acid moiety, a beta-amino acid moiety, or a gamma-amino acid moiety. In another aspect, L₂ is an alpha-amino acid derived moiety, a beta-amino acid derived moiety, or a gamma-amino acid derived moiety. In one aspect, L₂ is an oligoethylene oxide moiety. In one aspect, L₂ is (C1-C12)alkylenyl wherein one or more carbon atoms of the alkylenyl group can be substituted with (1) oxo or halo; (2) one or more carbon atoms can be replaced with an oxygen, sulfur, or nitrogen; or (3) two or three carbon atoms of the alkylenyl group can be taken to together to form a 4-, 5-, or 6-membered heterocyclic or cycloalkyl ring which can be optionally substituted, or a combination thereof. In one aspect, L₂ is an optionally substituted heterocyclyl or cycloalkyl moiety. In one aspect, L₂ comprises (1) an optionally substituted heterocyclyl or cycloalkyl moiety covalently linked to an (2) alkylenyl or amino acid or amino acid derivative moiety. In one aspect, L₂ comprises hydroxytetrahydrofuranyl. In one aspect, L₂ comprises 2-hydroxytetrahydrofuranyl or 3-hydroxytetrahydrofuranyl. In one specific implementation, L₂ comprises a 3-hydroxytetrahydrofuranyl group covalently linked to the remainder of the molecule through the 2- and 4-positions of the tetrahydrofuran ring. In one specific implementation, L₂ comprises an amino acid moiety derived from beta-alanine (e.g., —CH₂—CH₂—C(═O)—).

In another specific implementation, L₂ comprises an amino acid moiety derived from beta-alanine covalently linked to a 3-hydroxytetrahydrofuranyl moiety. In one aspect, L₂ comprises a sugar moiety. In one aspect, L₂ comprises a sugar moiety selected from a monosaccharide, disaccharide, oligosaccharide, polysaccharide, or a glycosaminoglycan. In one aspect, L₂ is

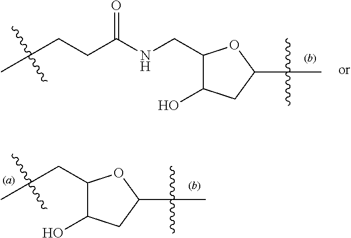 [SMILES: *CCC(=O)NCC1OC(*)CC1O ; *CC1OC(*)CC1O]

where bond (a) is bonded to (PAC) and bond (b) is bonded to Z.

If a metal sensor compound has more than one L₂ e.g., m is greater than 1, these groups can be chosen independently, that is to say, they can be different, or they can be the same.

The solubilizing moiety (X) in Formula (I) is covalently attached to (PAC) by the linker L₁. The term “solubilizing moiety” as defined herein is any group that enhances or increases the solubility of the compound having the Formula (I) in a particular solvent compared to the same compound that does not possess the solubilizing moiety in the same solvent. The number of solubilizing moieties can vary depending upon the application. In one aspect, the solubilizing moiety can facilitate the complete dissolution of the compound in the solvent. In one aspect, there can be 1, 2, 3, 4, 5, or 6 solubilizing moieties present in the compounds described herein. The solubilizing moiety is typically chosen based on the selection of the solvent the compound will be dissolved in. In one aspect, the solvent is an organic solvent. In another aspect, the solvent is water. Although the solubilizing moiety enhances the solubility of the compounds described herein, other groups present in the metal sensor compound can be selected to further enhance solubility. For example, L₁ and/or L₂ can be a hydrophilic group such as, for example, an oligoethylene oxide defined herein in order to increase the water-solubility of the compounds described herein.

The solubilizing moiety is chosen such that it does not render the selectivity or specificity of the metal ion binding properties of the metal sensor compounds described herein undesirable or unacceptable. Furthermore, the solubilizing moiety is chosen such that it does not render the absorbance, fluorescence or fluorescence quenching properties of the metal sensor compound undesirable or unacceptable. Desirably, the solubilizing moiety in combination with the remainder of the tunable metal sensor compound is chosen such that the compound is stable in selected solvents or solutions or as a solid. If the metal sensor compound has more than one solubilizing moiety, e.g., n is greater than 1 in Formula (I), the solubilizing moieties can be chosen independently, that is to say, they can be different, or they can be the same.

In one aspect, the solubilizing moiety is a pyridyl group, an alkyl pyridinium group (i.e., alkylation at nitrogen atom), a substituted pyridyl group, a substituted alkyl pyridinium group, or substituted phenyl group. In this aspect, the pyridyl or pyridinium group can be linked to (PAC) at the 2, 3, or 4 position of the pyridyl or pyridinium group. In some aspects, the alkyl group of the alkyl pyridinium can be a (C1-C8)alkyl. Examples of (C1-C8)alkyl groups include, but are not limited to, methyl, ethyl, propyl, isopropyl, butyl, n-butyl, isobutyl, and tert-butyl. In some aspect, the alkyl group of the alkyl pyridinium group is a methyl or ethyl group.

In another aspect, the solubilizing moiety is a phenyl group substituted with one or more groups that can increase the solubility of the compound is a particular solvent. In one aspect, the substituents can be a residue of an organic acid (e.g., —(CH₂)ₒCO₂H, where o is from 1 to 10)) or inorganic acids (e.g., —S(O)₂OH and —OP(O)₂OH)). In another aspect, the solubilizing moiety can be a polyalkyene oxide such as, for example, an oligoethylene oxide, a propylene oxide, or a block copolymer of ethylene oxide/propylene oxide). The molecular weight of the polyalkyene oxide can vary. In one aspect, the polyalkylene oxide can have from 1 to 20 ethylene oxide (—CH₂CH₂O—).

The metal ion binding ligand (Z) is covalently attached to the polycyclic aryl group via the linker, L₂, which together are designated (L₂-Z)ₘ in Formula (I). The variable m is an integer selected from 1, 2, 3, or 4, wherein m represents the number of metal binding ligands linked to the polycyclic aryl group.

In one aspect, Z is 1H-pyrimidine-2,4-dione or a derivative or analog thereof. Accordingly, one 1H-pyrimidine-2,4-dione derivative of this aspect is 5-methyl-1H-pyrimidine-2,4-dione. In one aspect, the 1H-pyrimidine-2,4-dione is substituted with one or two groups which are electron withdrawing groups or an electron donating group which alters the metal ion binding selectivity, specificity, or both, of the metal binding moiety of the metal sensor compound. Examples of electron withdrawing groups include, but are not limited to, a halo (e.g., —Br, —Cl, —F, or —I), haloalkyl (e.g., —CF₃, —CHF₂, or —CH₂F) and the like. Examples of electron donating groups include, but are not limited to, alkyl groups, like methyl, ethyl, propyl, isopropyl and the like. In one aspect, Z has the formula

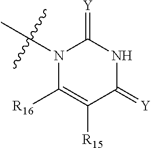

wherein each R₁₅ and R₁₆ is, independently, hydrogen, a halide, an alkyl group, or a haloalkyl group; and
each Y is, independently, oxygen or sulfur. In another aspect, each Y is oxygen. In a further aspect, each Y is oxygen and R₁₆ is hydrogen. In another aspect, each Y is oxygen, R₁₆ is hydrogen, and R₁₅ is methyl.

In another aspect, the metal binding ligand is a halo derivative of 1H-pyrimidine-2,4-dione is 5-fluoro-1H-pyrimidine-2,4-dione, 5-chloro-1H-pyrimidine-2,4-dione, 5-bromo-1H-pyrimidine-2,4-dione, or 5-iodo-1H-pyrimidine-2,4-dione. An alkyl derivative of 1H-pyrimidine-2,4-dione is 5-methyl-1H-pyrimidine-2,4-dione, 5-ethyl-1H-pyrimidine-2,4-dione, 5-propyl-1H-pyrimidine-2,4-dione or 5-butyl-1H-pyrimidine-2,4-dione. A haloalkyl derivative of 1H-pyrimidine-2,4-dione is 5-trifluoromethyl-1H-pyrimidine-2,4-dione, 5-difluoromethyl-1H-pyrimidine-2,4-dione, or 5-fluoromethyl-1H-pyrimidine-2,4-dione. In a preferred aspect, the 1H-pyrimidine-2,4-dione or a derivative or analog thereof is linked to the rest of the molecule through the nitrogen at the 1-position of the pyrimidine ring (as shown in Compounds 1, 2, 3, 4, and 5).

In one aspect, the metal sensor compound has the formula II, or a salt, solvate, or clathrate thereof, is provided as shown below:

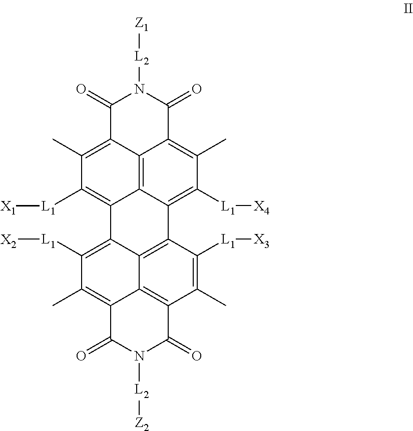

wherein

each L₁ independently is a linker that covalently connects the solubilizing moieties X₁, X₂, X₃, and X₄ to the polycyclic aryl group;

each L₂ independently is a linker that covalently connects a metal ion binding moiety Z₁ and Z₂, to the polycyclic aryl group;

each of X₁, X₂, X₃, and X₄ independently is a solubilizing moiety that is an optionally substituted heterocyclyl group or a substituted aryl group; and

each of Z₁ and Z₂ independently is a metal ion binding moiety that is 1H-pyrimidine-2,4-dione or a derivative or analog thereof.

In one aspect, L₁ is selected from alkylenyl, —O—, —NH—, —S— and oligoethylene oxide. In another aspect, L₁ is —O—. Each L₁ in Formula (II) can be chosen independently, that is to say they can be different, or they can be the same.

In some aspects of this embodiment, L₂ comprises an amino acid moiety or an amino acid derived moiety. In one aspect, L₂ comprises an alpha-amino acid moiety, a beta-amino acid moiety, or a gamma-amino acid moiety. In one aspect, L₂ comprises an alpha-amino acid derived moiety, a beta-amino acid derived moiety, or a gamma-amino acid derived moiety. In one aspect, L₂ comprises an oligoethylene oxide moiety. In one aspect, L₂ is (C1-C12)alkylenyl wherein one or more carbon atoms of the alkylenyl group can be substituted with (1) oxo or halo (e.g., a fluoroalkyl group having a —CF₂— group such as —CF₂CF₂—); (2) one or more carbon atoms can be replaced with an oxygen, sulfur, or nitrogen; or (3) two or three carbon atoms of the alkylenyl group can be taken to together to form a 4-, 5-, or 6-membered heterocyclic or cycloalkyl ring which can be optionally substituted, or a combination thereof. In one aspect, L₂ comprises an optionally substituted heterocyclyl or cycloalkyl moiety. In one aspect, L₂ comprises (1) an optionally substituted heterocyclyl or cycloalkyl moiety covalently linked to an (2) alkylenyl or amino acid or amino acid derivative moiety. In one aspect, L₂ comprises hydroxytetrahydrofuranyl. In one aspect, L₂ comprises 2-hydroxytetrahydrofuranyl, 3-hydroxytetrahydrofuranyl or 2,3-dihydroxytetrahydrofuranyl. In one specific implementation, L₂ comprises a 3-hydroxytetrahydrofuranyl group covalently linked to the remainder of the molecule through the 2- and 4-positions of the tetrahydrofuran ring. In one specific implementation, L₂ comprises an amino acid moiety derived from beta-alanine (e.g., —CH₂—CH₂—C(═O)—). In another specific implementation, L₂ comprises an amino acid moiety derived from beta-alanine covalently linked to a 3-hydroxytetrahydrofuranyl moiety. In one aspect, L₂ comprises a sugar moiety. In one aspect, L₂ comprises a sugar moiety selected from a monosaccharide, disaccharide, oligosaccharide, polysaccharide, or a glycosaminoglycan. In one aspect, L₂ has the formula

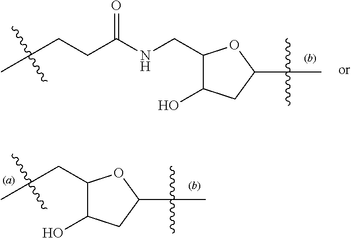 [SMILES: *CCC(=O)NCC1OC(*)CC1O ; *CC1OC(*)CC1O]

wherein bond (a) is bonded to (PAC) and bond (b) is bonded to the metal binding ligand Z.

Each L₂ in Formula (II) can be chosen independently, that is to say they can be different, or they can be the same.

In one aspect, each X₁ᵢ in Formula (II) is pyridyl, alkyl pyridinium, substituted pyridyl, substituted alkyl pyridinium, or substituted phenyl. In a more specific aspect, the pyridyl or pyridinium, or substituted derivative thereof, has the ring nitrogen at the 2-position in reference to the linker L₂. In another specific aspect, the pyridyl or pyridinium, or substituted derivative thereof, has the ring nitrogen at the 3-position in reference to the linker L₂. In another specific aspect, the pyridyl or pyridinium, or substituted derivative thereof, has the ring nitrogen at the 4-position in reference to the linker L₂. In some aspects, the alkyl group of the alkyl pyridinium can be a (C1-C8)alkyl. Examples of (C1-C8)alkyl groups include, but are not limited to, methyl, ethyl, propyl, isopropyl, butyl, n-butyl, isobutyl, and tert-butyl. In some aspect, the alkyl group of the alkyl pyridinium group is a methyl or ethyl group.

In another aspect, each X₁ᵢ is a phenyl group substituted with at least one hydrophilic group. In a more specific aspect, the phenyl group has a one, two, or three hydrophilic substituents. The hydrophilic group can be at the ortho, meta, or para position of the phenyl group relative to linker L₁. In one aspect, the hydrophilic group is —S(O)₃H (or salt thereof) and oligoethylene oxide. In one aspect, each phenyl group has one —S(O)₃H group.

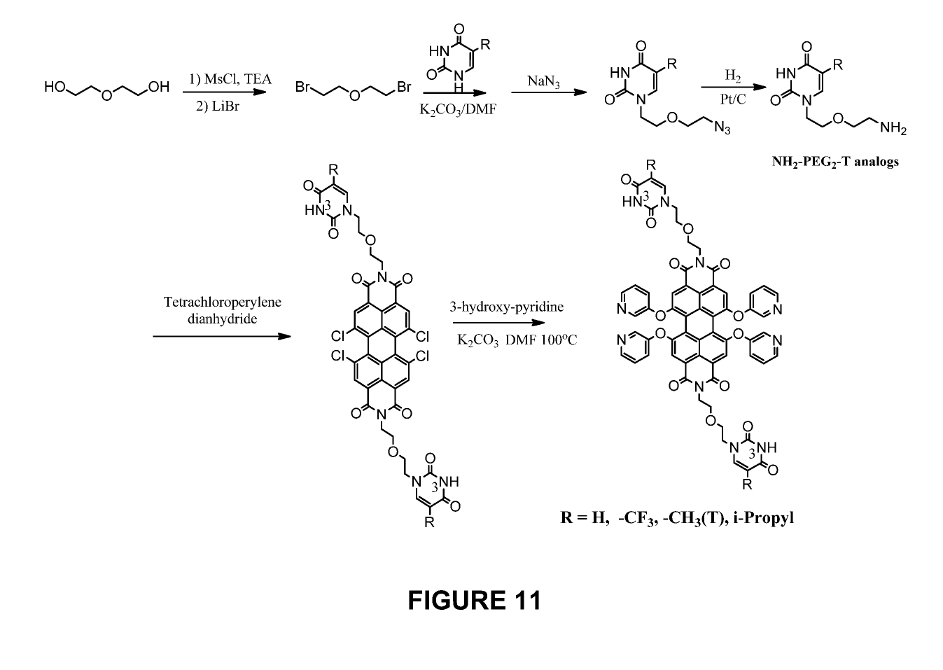

In one aspect, the binding ligand Z in Formula (II) is 1H-pyrimidine-2,4-dione or a derivative or analog thereof. Accordingly, one 1H-pyrimidine-2,4-dione derivative of this aspect is 5-methyl-1H-pyrimidine-2,4-dione. In one aspect, the 1H-pyrimidine-2,4-dione is substituted with one or two groups which are electron withdrawing groups or an electron donating group which alters the metal ion binding selectivity, specificity, or both, of the metal binding moiety of the metal sensor compound. Examples of electron withdrawing groups include, but are not limited to, a halo (e.g., —Br, —Cl, —F, or —I), haloalkyl (e.g., —CF₃, —CHF₂, or —CH₂F) and the like. Examples of electron donating groups include, but are not limited to, alkyl groups, like methyl, ethyl, propyl, isopropyl and the like. An exemplary procedure for making compounds having the Formula (II) where Z is 1H-pyrimidine-2,4-dione derivatives can be found in FIG. 11. In one aspect, each Z in Formula (II) has the formula

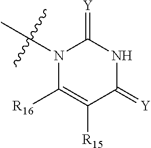

wherein each R₁₅ and R₁₆ is, independently, hydrogen, a halide, an alkyl group, or a haloalkyl group; and

each Y is, independently, oxygen or sulfur. In another aspect, each Y is oxygen. In a further aspect, each Y is oxygen and R₁₆ is hydrogen. In another aspect, each Y is oxygen, R₁₆ is hydrogen, and R₁₅ is methyl.

In one aspect, the metal binding ligand is a halo derivative of 1H-pyrimidine-2,4-dione is 5-fluoro-1H-pyrimidine-2,4-dione, 5-chloro-1H-pyrimidine-2,4-dione, 5-bromo-1H-pyrimidine-2,4-dione, or 5-iodo-1H-pyrimidine-2,4-dione. An alkyl derivative of 1H-pyrimidine-2,4-dione is 5-methyl-1H-pyrimidine-2,4-dione, 5-ethyl-1H-pyrimidine-2,4-dione, 5-prop yl-1H-pyrimidine-2,4-dione or 5-butyl-1H-pyrimidine-2,4-dione. A haloalkyl derivative of 1H-pyrimidine-2,4-dione is 5-trifluoromethyl-1H-pyrimidine-2,4-dione, 5-difluoromethyl-1H-pyrimidine-2,4-dione, or 5-fluoromethyl-1H-pyrimidine-2,4-dione. In a preferred aspect, the 1H-pyrimidine-2,4-dione or a derivative or analog thereof is linked to the rest of the molecule through the nitrogen at the 1-position of the pyrimidine ring (as shown in Compounds 1, 2, 3, 4, and 5).

Any of the compounds described herein can exist or be converted to the salt thereof. The salts can be prepared by treating the free acid with an appropriate amount of base. Representative bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. For example, when the solubilizing group is an acid such as —SO₃H, a base can be added to produce corresponding anion. In other aspects, when the compound possesses basic groups, the compound can be reacted with an acid or alkylating agent to produce the cationic salt. For example, when the solubilizing group is a pyridine group, the nitrogen atom can be protonated or alkylated to produce the cationic salt.

The term solvate comprises the solvent addition forms the compounds described herein. Examples of such solvent addition forms include, but are not limited to, hydrates, alcoholates and the like.

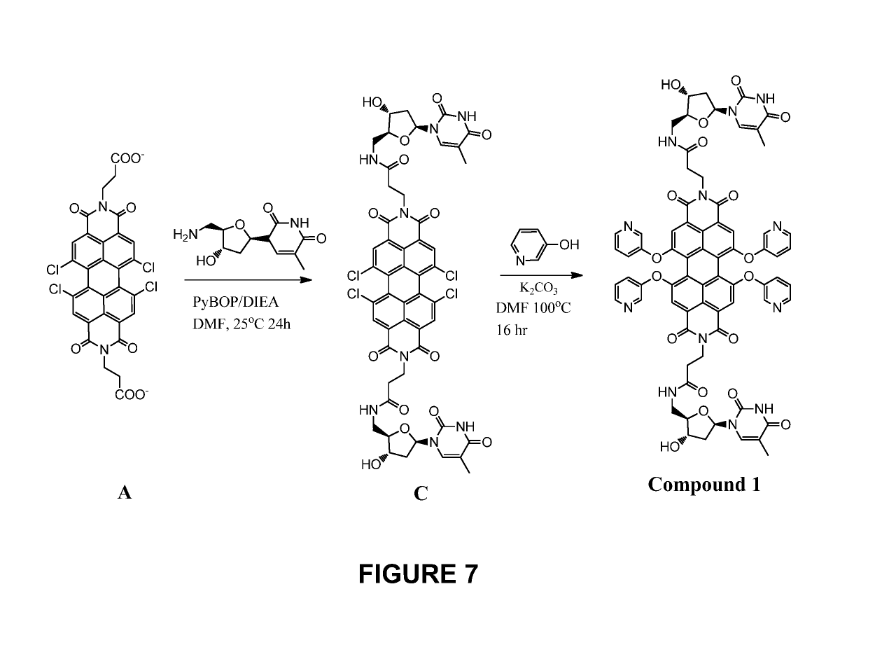

Methods for making the metal sensor compounds described herein are provided. For example, referring to FIG. 6, the synthesis starts from an appropriate PAC compound e.g., a tetrachloroperylene dianhydride or an analog or derivative thereof, which is coupled with two linkers (e.g., two molecules of β-alanine) to yield intermediate Compound A, or an analog thereof. Chloride displacement with a solubilizing group (e.g., 3-hydroxypyridine) (compound B) followed by condensation with a metal binding moiety (e.g., 5-methyl-1H-pyrimidine-2,4-dione) produces Compound 1. This procedure can be used in general to produce the metal sensor compounds described herein. Additionally, FIGS. 7-11 and the Examples provide exemplary procedures for making the metal sensor compounds described herein.

Applications

The compounds described herein selectively bind metal ions. In one aspect, the metal sensor compounds described herein bind selectively to one metal ion selected from Cu²⁺, Ni²⁺, Fe²⁺, Fe³⁺, Pb²⁺, Cd²⁺, Hg²⁺, Zn²⁺, Mn²⁺, Ba²⁺, Mg²⁺, Ca²⁺, Cr²⁺, Co²⁺, an ion of arsenic (As⁵⁺, As³⁺), antimony (Sb⁵⁺ or Sb³⁺), or thallium (Tl⁺ or Tl³⁺), a rare earth metal ion, a lanthanide metal ion, an actinide metal ion, or any combination thereof, while not binding selectively to the other metal ions. Selective binding refers to the metal ion causing a greater than 2-fold, 3-fold, 4-fold, 5-fold, 6-fold, 7-fold, 8-fold, 9-fold or 10-fold detectable spectral change e.g., in fluorescence, of the metal sensor compound as compared to the one or more, or 2, 3, 4, 5, 6, 7, 8, 9, or 10 or more of the other metal ions. In one aspect, the metal sensor compounds bind selectively to Hg²⁺.

In another aspect, a complex of a metal sensor compound and a metal ion is provided. The metal ion can be a metal cation. In one aspect, the metal cation is a divalent cation. In one aspect, the complex comprises 2 or more or 3, 4, 5, 6, 7, 8, 9, or 10 or more divalent cations. In one aspect, the complex comprises 2 or more or 3, 4, 5, 6, 7, 8, 9, or 10 or more metal sensor compounds. In one aspect, the metal ion is Hg²⁺. In one aspect, the complex comprising a metal sensor compound and a metal ion is produced by contacting a solution having one or more, or 2, 3, 4, or 5 or more different metal ions selected from Cu²⁺, Ni²⁺, Fe²⁺, Fe³⁺, Pb²⁺, Cd²⁺, Hg²⁺, Zn²⁺, Mn²⁺, Ba²⁺, Mg²⁺, Ca²⁺, Cr²⁺, Co²⁺, an ion of arsenic (As⁵⁺, As³⁺), antimony (Sb⁵⁺ or Sb³⁺), or thallium (Tl⁺ or Tl³⁺), a rare earth metal ion, a lanthanide metal ion, an actinide metal ion, or any combination thereof with the compound under conditions sufficient for forming a complex. In one aspect, the complex is a clathrate, i.e., a host-guest complex, where the metals sensor compound described herein entraps a metal ion (e.g, Hg²⁺).

In yet another aspect, a precipitate or aggregate comprising a metal sensor compound described herein and a metal ion is provided. In some aspects, the precipitate comprises a metal ion which is a metal cation. The precipitate can include a metal cation which is a divalent cation. In some aspects, the precipitate comprises 2 or more or 3, 4, 5, 6, 7, 8, 9, or 10 or more divalent cations. In one aspect, the precipitate comprises 2 or more or 3, 4, 5, 6, 7, 8, 9, or 10 or more metal sensor compounds. In one aspect, the precipitate comprises a metal ion which is Hg²⁺. In another aspect, the precipitate or aggregate comprises a metal sensor compound and a metal ion produced by contacting a solution having one or more, or 2, 3, 4, or 5 or more metal ions selected from Cu²⁺, Ni²⁺, Fe²⁺, Fe³⁺, Pb²⁺, Cd²⁺, Hg²⁺, Zn²⁺, Mn²⁺, Ba²⁺, Mg²⁺, Ca²⁺, Cr²⁺, Co²⁺, an ion of arsenic (As⁵⁺, As³⁺), antimony (Sb⁵⁺ or Sb³⁺), or thallium (Tl⁺ or Tl³⁺), a rare earth metal ion, a lanthanide metal ion, an actinide metal ion, or any combination thereof with the metal sensor compound under conditions sufficient for forming a precipitate or aggregate.

In another aspect, the invention relates to a method of fluorescing. The emission or fluorescence can occur at any wavelength of light longer than the excitation wavelength. Preferably, the emission or fluorescence occurs at a wavelength or wavelengths of light that are detectably different (longer) than the excitation wavelength or wavelengths. In some aspects, the peak fluorescence occurs within the range of 300 nm to 700 nm. In some aspects, the peak fluorescence occurs within the range of 300 nm to 500 nm. In some aspects, the peak fluorescence occurs within the range of 500 nm to 700 nm. In some aspects, the peak fluorescence occurs within the range of 500 nm to 600 nm. In some aspects, the peak fluorescence is at greater than 550 nm. The wavelength (or bandwidth) of light used for excitation or irradiation can be at any wavelength of light that the metal sensor compound absorbs light. In some aspects, the excitation wavelength is within the range of 180 nm to 700 nm. In some aspects, the excitation wavelength is within the range of 200 nm to 600 nm. In some aspects, the excitation wavelength is within the range of 210 nm to 550 nm. In some aspects, the excitation or detection wavelength(s) are controlled by a short pass or long pass filter, respectively.

In another aspect, described herein is a method of quenching fluorescence of a metal sensor compound described herein. According to this aspect, the method involves contacting the metal sensor compound with a metal ion that quenches the fluorescence of the metal sensor compound. In one aspect, the quenching of fluorescence, or lack thereof, is determined by contacting a test sample suspected of having a metal ion or that is desired to be tested for a metal ion with a metal sensor solution comprising a metal sensor compound and determining the fluorescence of the test sample solution. In some aspects, the method involves comparing the test sample solution fluorescence to the fluorescence of a metal sensor solution comprising a metal sensor compound that was not contacted with a metal ion or contacted with a vehicle not containing a metal ion (e.g., metal sensor solution). Here, a difference in fluorescence indicates the metal ion is present in the test sample.

In other aspects, the metal sensor compounds described herein can be used to determine the metal ion concentration in a test sample. In one aspect, the method for quantifying a metal ion concentration in a test sample involves:

comprising contacting the test sample with a calibration solution comprising a known concentration of metal sensor compound described herein and measuring the fluorescence of the test sample;

calibrating the fluorescence of the test sample to a calibration curve to determine the concentration of the metal ion in the test sample.

In this aspect, the method involves calibrating with a test calibration solution (i.e., a solution having a known concentration of metal sensor compound) to determine metal ion concentration. In some aspects, the fluorescence is quenched by greater than 1% by addition of metal ion to the metal sensor solution. Here, a calibration curve can be used to quantify the amount of metal ion is present in the test sample based on the level of fluorescence that is quenched by the metal ion. In some aspects, the fluorescence is quenched by greater than 25% by addition of metal ion to the metal sensor solution. In some aspects, the fluorescence is quenched by greater than 50% by addition of metal ion to the metal sensor solution. In some aspects, the fluorescence is quenched by greater than 75% by addition of metal ion to the metal sensor solution. In some aspects, the fluorescence is quenched by greater than 90% by addition of metal ion to the metal sensor solution. The quenching of fluorescence can be rapid or slow. In some implementations a rapid quenching of fluorescence is desirable. Thus, in one aspect of this embodiment, quenching of 1% or more, 5% or more, 10% or more, 25% or more, 50% or more, 75% or more, 90% or more, or 95% or more, occurs within 1 hour of exposure of a metal sensor solution to a test sample having a metal ion, or 45 minutes, or 30 minutes, or 10 minutes, or 5 minutes or less.

In another aspect, a method for determining a difference in fluorescence is provided. In one aspect, the method involves contacting a metal sensor solution comprising the metal sensor compound with a test sample and determining a difference in fluorescence as compared to the fluorescence of a metal sensor solution comprising the metal sensor compound not having a test sample. In one aspect, the method for determining a difference in fluorescence, or lack thereof, comprises (a) contacting a test sample suspected of having a metal ion or that is desired to be tested for a metal ion with a solution comprising the metal sensor compound, (b) determining the fluorescence of the solution and (c) comparing to the fluorescence of a solution comprising the metal sensor compound that was not contacted with a metal ion or was contacted with a vehicle not having a metal ion wherein the test sample has a concentration of one or more, or 2, 3, 4, or 5 or more different metal ions selected from Cu²⁺, Ni²⁺, Fe²⁺, Fe³⁺, Pb²⁺, Cd²⁺, Hg²⁺, Zn²⁺, Mn²⁺, Ba²⁺, Mg²⁺, Ca²⁺, Cr²⁺, Co²⁺, an ion of arsenic (As⁵⁺, As³⁺), antimony (Sb⁵⁺ or Sb³⁺), or thallium (Tl⁺ or Tl³⁺), a rare earth metal ion, a lanthanide metal ion, an actinide metal ion, or any combination thereof each at a concentration of greater than 1, 2, 3, 4, 5, 10, 15, 20, 25, 50, 75, 100, 150, 200, 300, 400, or 500 ppb. In another aspect, the method comprises a before and after reading of fluorescence where a measurement of fluorescence of the test sample is made before addition of a metal sensor solution comprising the metal sensor compound is made and another measurement of fluorescence is made after addition of a metal sensor solution comprising the metal sensor compound thereby determining a difference in fluorescence.

In one aspect, the compounds described herein are useful in detecting and quantifying metal ions (e.g., Hg²⁺) in a fluid. The term “fluid” as used herein is any liquid or gas that contains metal ions. Examples of fluids that contain mercury ion include, but are not limited to, water, an aqueous based solution, air, hydrogen, natural gas, exhaust gas from the thermal destruction of chemical warfare munitions, liquid hydrocarbons, wastewater discharge chlor-alkali plant, waste streams from dye, pharmaceutical, and agrochemical manufacturing, waste streams from mining, waste streams from concrete or cement production, waste streams from production of printed circuit boards or other electroplating processes, waste generated during thermometer or vacuum pump gauge manufacturing, exhaust gas passed through a water scrubber, an air stream produced from an air purification system, waste materials generated from the production of nuclear weapons, or an offgas from (1) a mixed waste incineration, (2) a plasma enhanced melter, and (3) ventilation of a hot cell. In other aspects, the fluid is municipal water supplies, personal & domestic water supplies, river water, lake water, pond water, groundwater, or a soil extract. In other aspects, the fluid is a dyestuff or pharmaceutical ingredient.

In some aspects, the methods described herein involve additional processing of the test sample prior to contacting with the sample with the metal sensor compound. For example, it may be advantageous to filter a test sample prior to use in the methods described herein. Another example of processing can include a method for preparing inorganic metal ions from an organic source. For example, mercury often exists in an organic form like methylmercury (HgCH₃⁺) in nature and the method can involve oxidizing methylmercury or other organic forms of mercury to Hg²⁺, thereby facilitating its detection/measurement as described herein. Any method that can convert a metal to a form compatible with the metal sensor compound and detection system may be used.

Mercury dissolved organic matter (DOM) and particulate organic matter (POM) or inorganic mercury may be converted to a form of mercury compatible with the methods and compounds described herein. Inorganic samples, include, but are not limited to, samples such as sand, soil, sediments etc. It may be that Hg²⁺ is simply physical absorbed on the surfaces of pores of the components of such samples. Ultrasound or dilute HNO₃ treatment may release Hg²⁺ to solution. After neutralization with base and filtration, Hg²⁺ in the filtrate can be detected by the compounds and method disclosed herein. If high amounts of Hg(O) or Hg(I) are assumed to be in a sample, it can treated in a manner similar to that described below for organic samples.

In one aspect, when the organic sample is DOM, POM, and animal tissues, the EPA recommends HNO₃/H₂SO₄ (7:3) for total digestion of organic samples at 80° C. for 6 hrs. The sample is next treated with 1% BrCl or H₂SO₄/HNO₃/KMnO₄ to oxidize mercury (O) and methyl mercury (I) to mercury (II) for 12 hr. The oxidation is quenched by NH₂OH.HCl. In some aspects, the methods may involve digestion and oxidization of the samples under mild conditions. Furthermore, it is contemplated that the Hg²⁺ solution can be neutralized with bases such as NaHCO₃, Na₂CO₃ to pH 6-8. It is contemplated that one or more of these procedures or similar procedures may be used to generate Hg²⁺ from samples containing other forms of mercury.

Prior to use, the metal sensor compounds are generally formulated in a solution. In one aspect, a solution comprising a solvent and a metal sensor compound described herein is provided. The solvent used herein for the solution comprising the metal sensor compound can have one component e.g., water, an organic solvent, or a combination thereof. In another aspect, the solvent is a mixture of water and one or more water-miscible organic solvents. Examples of water-miscible organic solvents useful herein include, but are not limited to, alkanols, glycols (e.g., diols like ethylene glycol or triols like glycerol) dimethylsulfoxide, dimethylformamide, dimethylacetamide, dichloromethane, acetone, dioxane, or tetrahydrofuran. Depending on the application, different solvents or solvent systems can be used in conjunction with the compositions and methods described herein.

In one aspect, a solution comprising a solvent and the metal sensor compound is provided wherein the solvent is an aqueous solvent. The aqueous solvents as described herein can comprise 2% or more H₂O, 5% or more H₂O, 10% or more H₂O, 20% or more H₂O, 30% or more H₂O, 40% or more H₂O, 50% or more H₂O, 60% or more H₂O, 70% or more H₂O, 80% or more H₂O, 90% or more H₂O, or 95% or more H₂O. In some aspects, the aqueous solvent comprises 2% or more glycol, 5% or more glycol, 10% or more glycol, 20% or more glycol, 30% or more glycol, 40% or more glycol, 50% or more glycol. In some aspects, the glycol is glycerol. In other aspects, the glycol is propylene glycol.

In one aspect, the metal sensor compounds described herein can be admixed with a water miscible, non-volatile organic polymer, wherein the metal sensor compound is incorporated in the water miscible, non-volatile organic polymer. Examples of water miscible, non-volatile organic polymers useful herein include, but are not limited to, a polyalkylene alcohol (e.g., polyethylene glycol), a poloxamer, polyvinyl alcohol, polyvinylpyrrolidone, poly (N-vinyl lactam), a polyacrylamide, a polyanhydride, a polyacrylic acid, a polyvinyl ether, polyethyleneimine, cellulose or a derivative thereof, or any combination

In some aspects, the aqueous solvent may further comprise a buffer. The buffer for use in these aspects is chosen such that it does not unacceptably or undesirably interfere with properties of the metal sensor compound. One buffer that can be used is an acetate buffer. For example, the buffer should not substantially bind to the metal ions for which the metal sensor compound was designed or should not interfere substantially with the ability of the tunable metal sensor compound to bind a selected metal ion or unacceptably or undesirably affect the spectral properties of the tunable metal sensor compound. If the buffer is used with a test solution for testing a sample for metal ions it should be present in such concentrations in the test solution as to prevent substantial changes in the pH of the test solution upon addition of a test sample thereto. For example a change in pH of 0.5 units, 1.0 or more units, or 2.0 or more units of the test solution upon addition of the test solution may affect the metal binding properties of the tunable sensor compound as described herein and therefore be unacceptable. One example of a suitable buffer for some implementation is an acetate based buffer.

In certain aspects, the test sample solution or test calibration solution is at a specific pH or within a pH range. According to some aspects, the test sample is adjusted to a pH or within a pH range described in this paragraph prior to addition of metal sensor solution. The test sample solution or test calibration solution is some aspects can be adjusted to the particular values disclosed in this paragraph by addition of a buffer or concentrated buffer solution, or acid or base. In some aspects, the test sample solution is at a pH between 3.0 and 8.5. In some aspects, the test sample solution is at pH 3.5 plus or minus 0.5. In some aspects, the test sample solution is at pH 4.0 plus or minus 0.5. In some aspects, the test sample solution is at pH 4.5 plus or minus 0.5. In some aspects, the test sample solution is at pH 5.0 plus or minus 0.5. In some aspects, the test sample solution is at pH 5.5 plus or minus 0.5. In some aspects, the test sample solution is at pH 6.0 plus or minus 0.5. In some aspects, the test sample solution is at pH 6.5 plus or minus 0.5. In some aspects, the test sample solution is at pH 7.0 plus or minus 0.5. In some aspects, the test sample solution is at pH 7.5 plus or minus 0.5. In some aspects, the test sample solution is at pH 8.0 plus or minus 0.5. In some aspects, the test calibration solution is at a pH between 3.0 and 8.5. In some aspects, the test calibration solution is at pH 3.5 plus or minus 0.5. In some aspects, the test calibration solution is at pH 4.0 plus or minus 0.5. In some aspects, the test calibration solution is at pH 4.5 plus or minus 0.5. In some aspects, the test calibration solution is at pH 5.0 plus or minus 0.5. In some aspects, the test calibration solution is at pH 5.5 plus or minus 0.5. In some aspects, the test calibration solution is at pH 6.0 plus or minus 0.5. In some aspects, the test calibration solution is at pH 6.5 plus or minus 0.5. In some aspects, the test calibration solution is at pH 7.0 plus or minus 0.5. In some aspects, the test calibration solution is at pH 7.5 plus or minus 0.5. In some aspects, the test calibration solution is at pH 8.0 plus or minus 0.5.

In one aspect, the metal sensor compound is present in the solution at a concentration in the range of 1 pM to 1 mM. In one aspect, the metal sensor compound is present in the solution at a concentration in the range of 0.1 nM to 300 μM. In one aspect, the metal sensor compound is present in the solution at a concentration in the range of 0.1 nM to 100 μM. In another aspect, the metal sensor compound is present in the solution at a concentration in the range of 0.1 nM to 50 μM.

The solution comprising the metal sensor compounds as described herein can be a storage or stock solution that is intended to be transferred to a test solution or it can be a test solution which is intended to be used for determining the presence, absence or quantity and type of metal ions in a solution. In one aspect, the solvent is dimethylformamide (DMF)-H₂O (about 7:3), ethanol-dichloromethane (DCM) (about 5:1), H₂O, ethanol, isopropanol, about 15% glycerol-H₂O, about 30% glycerol-H₂O, and about 50% glycerol-H₂O.

### EXAMPLES

The following examples are included to illustrate concepts and particular embodiments related to the invention. As will be appreciated by those of skill in the art, the techniques, methods and compositions disclosed in the following examples are representative of particular modes for practice of the invention while not being intended to limit scope of the invention.

### Example 1

### Synthesis of Tunable Metal Ion Sensing Compounds

The compounds provided herein e.g., of Formula (I) or (II) may be synthesized by a variety of procedures including, but not limited to, Route A (FIG. 6) and Route B (FIG. 7) as described below in more detail for the synthesis of Compound 1. Similar or analogous synthetic schemes using similar or different reagents, starting materials, reaction conditions, catalysts, and/or reactions may be employed by the skilled artisan to prepare various compounds of Formula (I) or (II). Title compounds identified with letters such as Compound A, Compound B, Compound C, etc., are key intermediate compounds. Title compounds identified with numbers such as Compound 1, Compound 2, Compound 3, etc., are metal sensor compounds or are additional compounds that can be used in industry in a manner similar to the way other perylene or perylene-3,4,9,10-tetracarboxylic diimides (or analogs or derivatives thereof) are used. Some of the compounds of Formula (I) and (II) may have properties that make them suited for uses other than metal sensing.

### Synthetic Route A (FIG. 6)

### Compound A: 3,3′-(5,6,12,13-tetrachloro-1,3,8,10-tetraoxoanthra[2,1,9-def:6,5,10-d′e′f′]diisoquinoline-2,9(1H,3H,8H,10H)-diyl)dipropanoic acid

A reaction mixture containing 5,6,12,13-tetrachloroanthra[2,1,9-def:6,5,10-d′e′f′]diisochromene-1,3,8,10-tetraone (1 g, 1.89 mmol) and β-alanine (370 mg, 4.16 mmol) in 15 mL of pyridine was stirred at 90° C. overnight. The mixture was concentrated to remove solvent and the residue was suspended into 30 mL of MeOH-water (1:1). Filtration and the solid was washed with acetone/MeOH to dryness. 1.15 g (91%) of brown solid was obtained. ESI (negative) 672.0 (M−H)⁻.

### Compound B: 3,3′,3″,3′″-((2,9-bis(2-carboxyethyl)-1,3,8,10-tetraoxo-1,2,3,8,9,10-hexahydroanthra[2,1,9-def:6,5,10-d′e′f′]diisoquinoline-5,6,12,13-tetrayl)tetrakis(oxy))tetrakis(pyridine-1-ium) chloride

A reaction mixture containing Compound A (0.2 g, 0.29 mmol), 3-Hydroxy-pyridine (216 mg, 2.38 mmol), K₂CO₃ (330 mg, 2.38 mmol) in 10 mL of DMF was heated at 100° C. overnight. The mixture was treated with 0.5 mL of HCl (12 M) and concentrated to remove solvents. The residue was purified by combiflash (12 g, DCM to 30% MeOH/DCM) afforded 30 mg (11%) of Compound B HCl. ¹HNMR (400 MHz, MeOH-d₄) 8.31 (s, 4H), 8.21 (s, 4H), 8.15 (s, 4H), 7.31 (s, 4H), 7.30 (s, 4H), 2.64 (m, 4H) ESI (Negative): 905.3 (M−H)⁻

### Compound 1: N-(((2R,3S,5R)-3-hydroxy-5-(5-methyl-2,4-dioxo-3,4-dihydropyrimidin-1(2H)-yl)tetrahydrofuran-2-yl)methyl)-3-(9-(3-((((2S,3R,5S)-3-hydroxy-5-(5-methyl-2,4-dioxo-3,4-dihydropyrimidin-1(2H)-yl)tetrahydrofuran-2-yl)methyl)amino)-3-oxopropyl)-1,3,8,10-tetraoxo-5,6,12,13-tetrakis(pyridin-3-yloxy)-9,10-dihydroanthra[2,1,9-def:6,5,10-d′e′f′]diisoquinolin-2(1H,3H,8H)-yl)propanamide

To the reaction mixture of Compound B HCl (15 mg, 0.0166 mmol), amino-thymidine (12 mg, 0.0415 mmol), DIEA (58 μL) in 1 mL of DMF was added PyBOP (35 mg, 0.0644 mmol). The suspended solution was stirred at room temperature overnight. DMF was removed and the residue was purified by combiflash (DCM to 30% MeOH/DCM) to give Compound 1, 13 mg (58%). ¹HNMR (400 MHz, MeOH-d₄) 8.30 (m, 8H), 8.03 (s, 4H), 7.72 (d, J=6 Hz, 4H), 7.61 (d, J=7.2 Hz, 4H), 7.35 (s, 4H), 6.11 (t, J=2.4 Hz, 2H), 4.58 (m, 2H), 4.26 (m, 2H), 4.19 (m, 2H), 3.99 (m, 2H), 3.83 (d, J=8.4 Hz, 2H), 3.52 (m, 2H), 2.31 (m, 2H), 1.72 (s, 2CH₃), ESI: 677.1 (M+2H)²⁺.

### Route B (FIG. 7)

### Compound C: N-(((2S,3R,5S)-3-hydroxy-5-(5-methyl-2,4-dioxo-3,4-dihydropyrimidin-1(2H)-yl)tetrahydrofuran-2-yl)methyl)-3-(5,6,12,13-tetrachloro-9-(3-((((2R,3S,5R)-3-hydroxy-5-(5-methyl-2,4-dioxo-3,4-dihydropyrimidin-1(2H)-yl)tetrahydrofuran-2-yl)methyl)amino)-3-oxopropyl)-1,3,8,10-tetraoxo-9,10-dihydroanthra[2,1,9-def:6,5,10-d′e′f′]diisoquinolin-2(1H,3H,8H)-yl)propanamide

To the reaction mixture of Compound A (63.3 mg, 0.0942 mmol), amino-thymidine (50 mg, 0.2072 mmol), DIEA (164 μL) in 2 mL of DMF was added PyBOP (196 mg, 0.0377 mmol). The suspended solution was stirred at room temperature overnight. DMF was removed and the residue was purified by combiflash (DCM to 30% MeOH/DCM) to give Compound C, 26.3 mg (25%). ¹HNMR (400 MHz, MeOH-d₄) 8.64 (s, 4H), 7.33 (m, 2H), 6.03 (m, 2H), 4.50 (s, 4H), 4.18 (m, 4H), 3.87 (m, 2H), 3.47 (m, 2H), 2.68 (m, 4H), 1.87 (s, 2CH₃), ESI: 560.0 (M+2H)²⁺′ 1118.8 (M+H)⁺.

### Compound 1: N-(((2R,3S,5R)-3-hydroxy-5-(5-methyl-2,4-dioxo-3,4-dihydropyrimidin-1(2H)-yl)tetrahydrofuran-2-yl)methyl)-3-(9-(3-((((2S,3R,5S)-3-hydroxy-5-(5-methyl-2,4-dioxo-3,4-dihydropyrimidin-1(2H)-yl)tetrahydrofuran-2-yl)methyl)amino)-3-oxopropyl)-1,3,8,10-tetraoxo-5,6,12,13-tetrakis(pyridin-3-yloxy)-9,10-dihydroanthra[2,1,9-def:6,5,10-d′e′f′]diisoquinolin-2(1H,3H,8H)-yl)propanamide

A reaction mixture containing Compound C (20 mg, 0.018 mmol), 3-Hydroxy-pyridine (13.6 mg, 0.143 mmol), K₂CO₃ (19.8 mg, 0.143 mmol) in 1 mL of DMF was heated at 100° C. overnight. The mixture was concentrated to remove solvents. The residue was purified by combiflash (4 g, DCM to 30% MeOH/DCM) afforded 5.3 mg (22%) of Compound 1. ¹HNMR (400 MHz, MeOH-d₄) 8.31 (s, 4H), 8.21 (s, 4H), 8.15 (s, 4H), 7.31 (s, 4H), 7.30 (s, 4H), 2.64 (m, 4H) ESI (Negative): 905.3 (M−H)⁻.

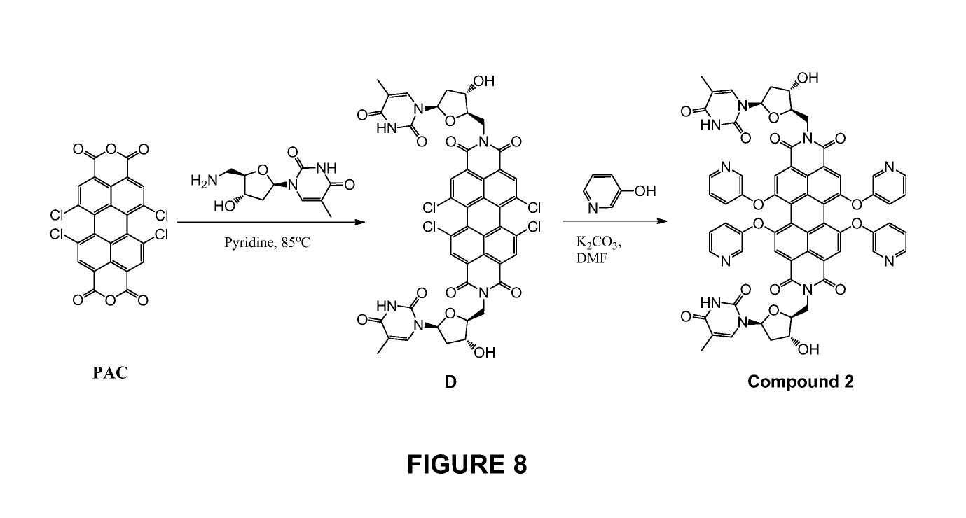

### Synthetic Route A (FIG. 8)

### Compound D: 5,6,12,13-tetrachloro-2-(((2R,3S,5R)-3-hydroxy-5-(5-methyl-2,4-dioxo-3,4-dihydropyrimidin-1(2H)-yl)tetrahydrofuran-2-yl)methyl)-9-(((2S,3R,5S)-3-hydroxy-5-(5-methyl-2,4-dioxo-3,4-dihydropyrimidin-1(2H)-yl)tetrahydrofuran-2-yl)methyl)anthra[2,1,9-def:6,5,10-d′e′f′]diisoquinoline-1,3,8,10(2H,9H)-tetraone

A reaction mixture containing 5,6,12,13-tetrachloroanthra[2,1,9-def:6,5,10-d′e′f′]diisochromene-1,3,8,10-tetraone (50 mg, 0.094 mmol) and amino-thymidine (50 mg, 0.207 mmol) in 2 mL of pyridine was stirred at 85° C. for 2 hrs. The mixture was concentrated to remove solvent and the residue was purified on SiO₂ combiflash (12 g, DCM to 30% MeOH/DCM) to give orange solid 53 mg (yield 58%). ¹HNMR (400 MHz, MeOH-d₄/CDCl₃): 8.64 (s, 4H), 7.69 (s, 2H), 6.25 (m, 2H), 4.37 (m, 4H), 2.23 (m, 6H), 2.07 (s, 6H). ESI: 976.8 (M+H)⁺.

### Compound 2: 2-(((2R,3S,5R)-3-hydroxy-5-(5-methyl-2,4-dioxo-3,4-dihydropyrimidin-1(2H)-yl)tetrahydrofuran-2-yl)methyl)-9-(((2S,3R,5S)-3-hydroxy-5-(5-methyl-2,4-dioxo-3,4-dihydropyrimidin-1(2H)-yl)tetrahydrofuran-2-yl)methyl)-5,6,12,13-tetrakis(pyridin-3-yloxy)anthra[2,1,9-def:6,5,10-d′e′f′]diisoquinoline-1,3,8,10(2H,9H)-tetraone

A reaction mixture containing Compound D (20 mg, 0.02 mmol), 3-Hydroxy-pyridine (15.7 mg, 0.164 mmol), K₂CO₃ (22.6 mg, 0.164 mmol) in 2 mL of DMF was heated at 100° C. for 3 hrs. The mixture was treated with HOAc and concentrated to remove solvents. The residue was purified by SiO₂ combiflash twice (4 g, DCM to 30% MeOH/DCM) afforded 3.1 mg (yield 12.5%) of Compound 2. ¹HNMR (400 MHz, MeOH-d₄) 8.36 (s, 4H), 8.27 (s, 4H), 8.15 (s, 4H), 7.7 (m, 4H), 7.46 (s, 4H), 7.40 (s, 4H), 6.26 (m, 2H), 4.36 (m, 4H), 4.12 (m, 2H), 3.66 (m, 2H), 2.04 (s, 3H) ESI: 606.2 (M+2H)²⁺.

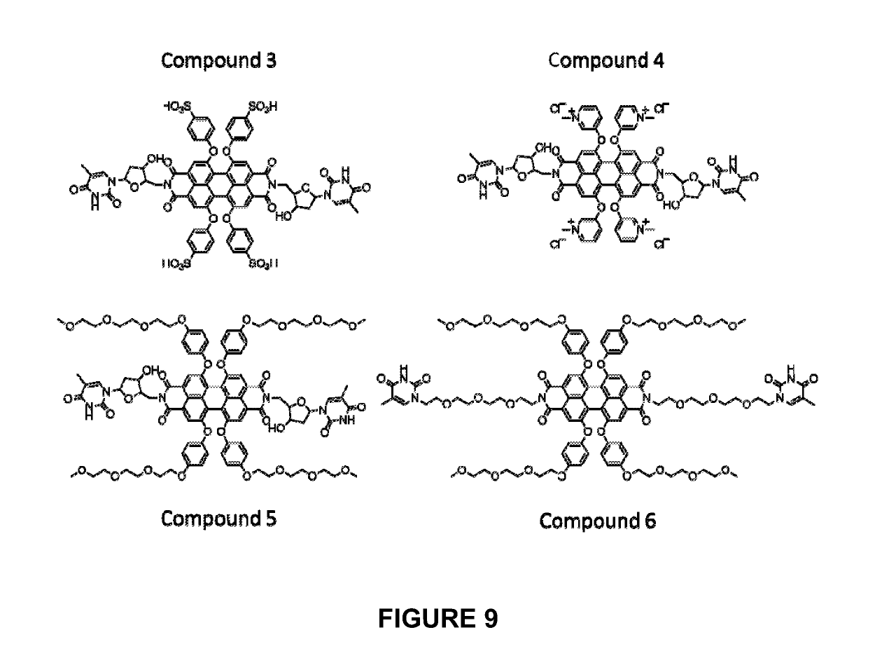

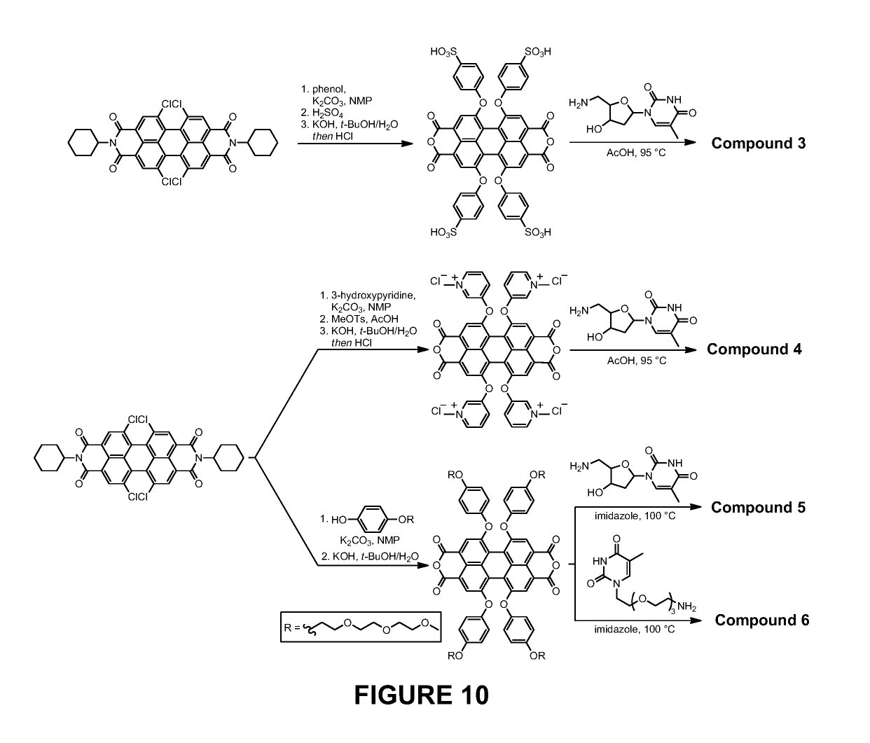

Synthesis of the compounds of Formula (I) or (II) can be accomplished following the methods disclosed herein and other established protocols through e.g., the common intermediate tetrachloroperylene cyclohexyldiimide. Briefly, chloride displacement with the appropriate phenol followed by imide hydrolysis, acidic workup, and subsequent condensation of the resulting dianhydride with either 5′-amino-5′-deoxythymidine will afford compounds of interest. Pyridine N-methylation in the presence of methyl p-toluenesulfonate (MeOTs), followed by a synthetic sequence analogous to that described above will afford an alkyl pyridinium target compound as shown in FIG. 9 (e.g., Compound 4). See FIG. 10 for additional details for preparing metal sensor Compounds 3-6.

### Example 2

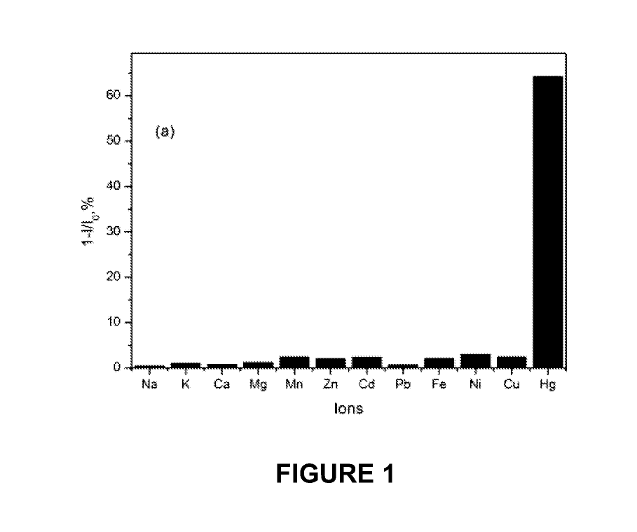

Fluorescence response of Compound 1 (1.0 μM) to Hg²⁺ (2.0 μM) and 11 other metal ions (5 μM each, Cu²⁺, Ni²⁺, Fe²⁺, Pb²⁺, Cd²⁺, Zn²⁺, Mn²⁺, Mg²⁺, Ca²⁺, K⁺, Na⁺) in pH 5 NaOAc solution (0.02 M). The bars represent the percentage of fluorescence quenched (1−I/I₀) %. See FIG. 1.

### Example 3

### Linearity of Detection Range

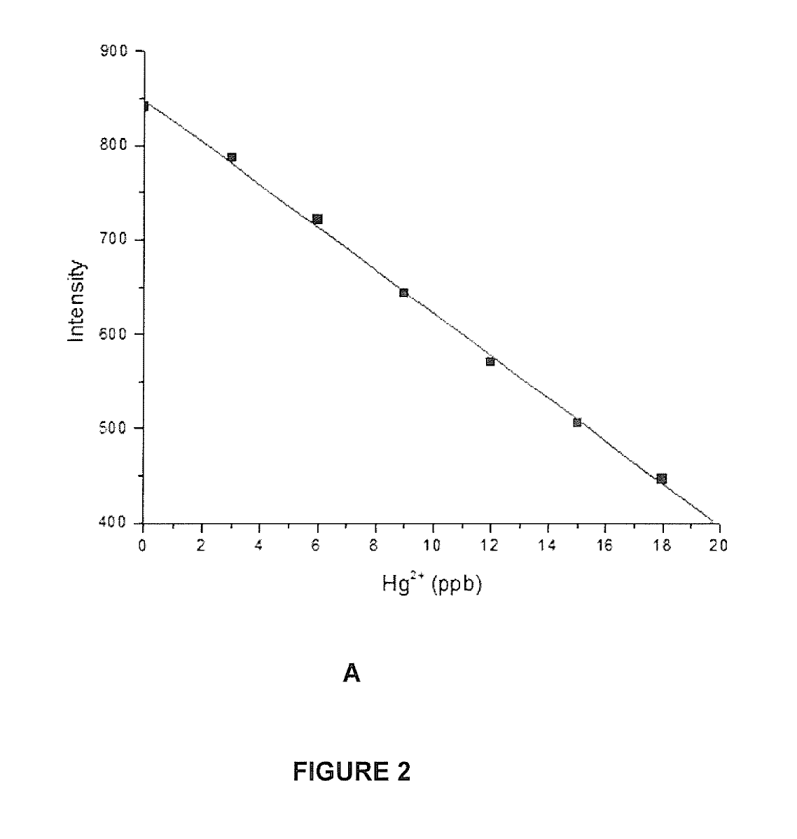

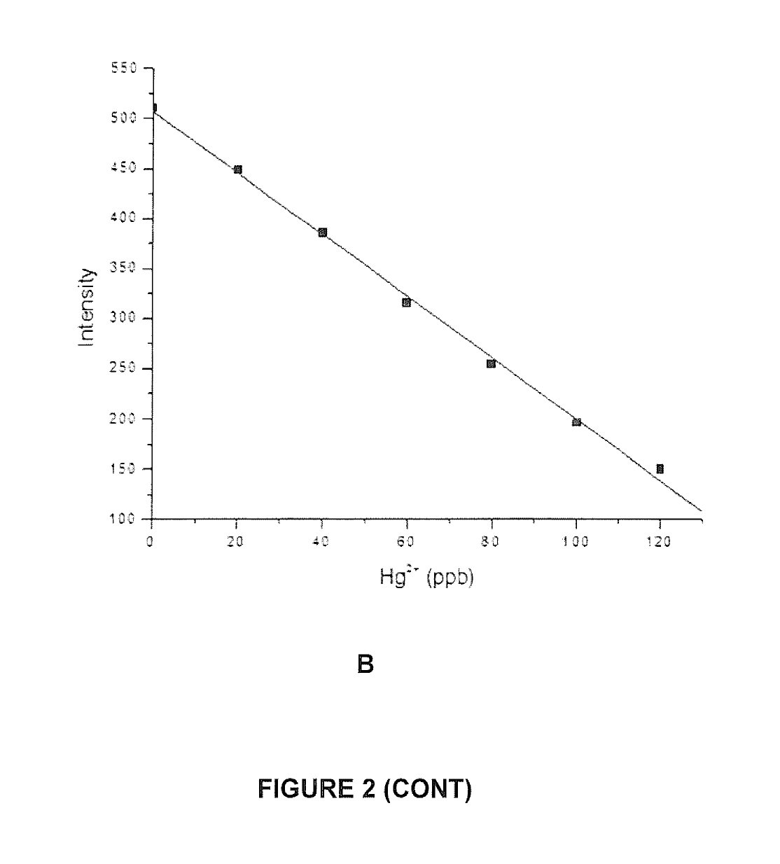

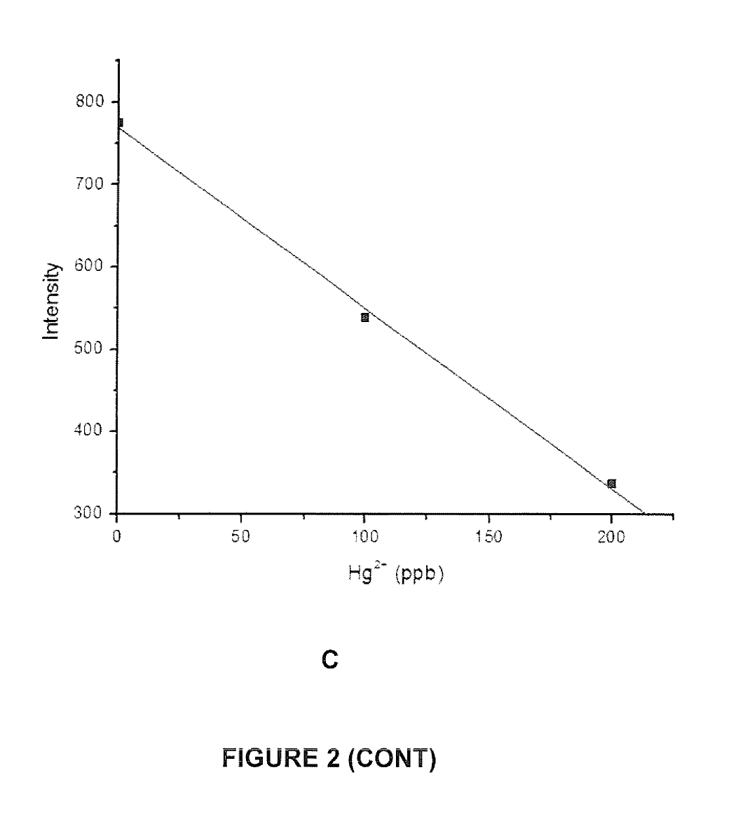

Linear detection response for Hg²⁺ was determined/established for several concentrations of Compound 1. A linear detection response of Hg²⁺ in the range 0 to 18 ppb for 0.3 μM of Compound 1 and a detection limit of 0.4 ppb for Hg²⁺, corresponding to a 1% decrease of fluorescence intensity was determined (FIG. 2A). FIG. 2B shows a linear detection response for Hg²⁺ in the range 0 to 120 ppb for 2 μM of Compound 1. FIG. 2C shows a linear detection response for Hg²⁺ in the range 0 to 200 ppb for 5 μM of Compound 1.

### Example 4

### Stability of Compound 1

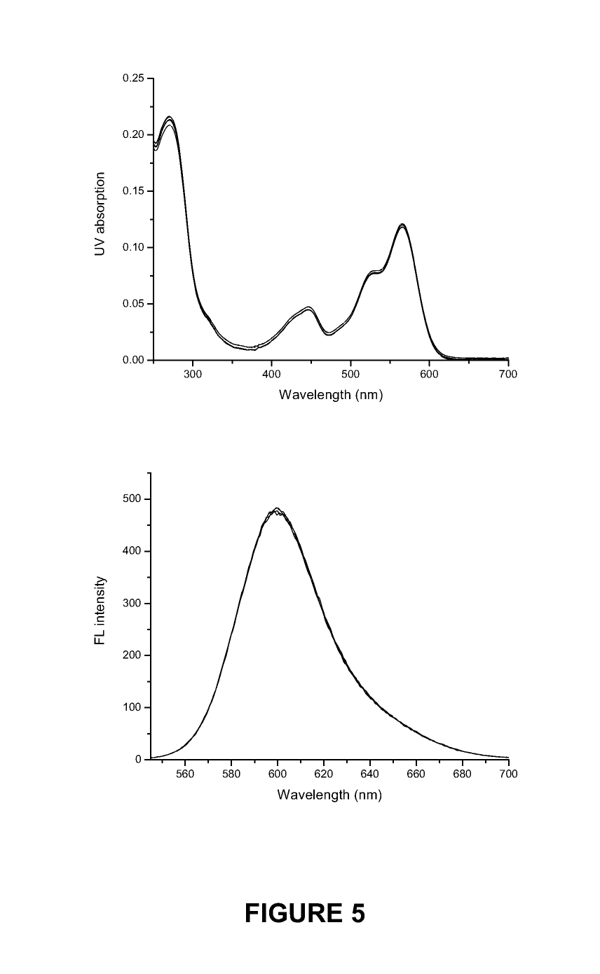

High density polyethylene (HDPE) and low density polyethylene (LDPE) squeeze bottles were used to store metal sensor solutions (7.4 μM Compound 1). One pair of HDPE and LDPE bottles was stored on shelf in room, and the other pair of bottles was stored outside in shade. Compound 1 is very stable in 50% glycerol-water without obvious change of absorbance and fluorescence spectra at least for 6 weeks. See FIG. 5 which shows the absorbance spectrum at day 0, 7, 15, and 20 of the metal sensors solutions stored in LPDE outside and the fluorescence spectrum at days 7, 14, and 20 of metal sensor solutions stored inside and outside in both LDPE and HDPE containers.

### Example 5

### pH Tuning Studies

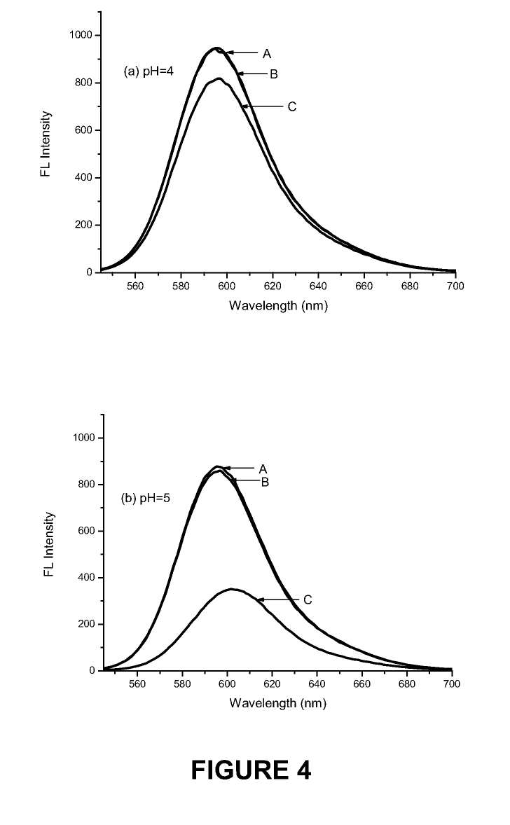

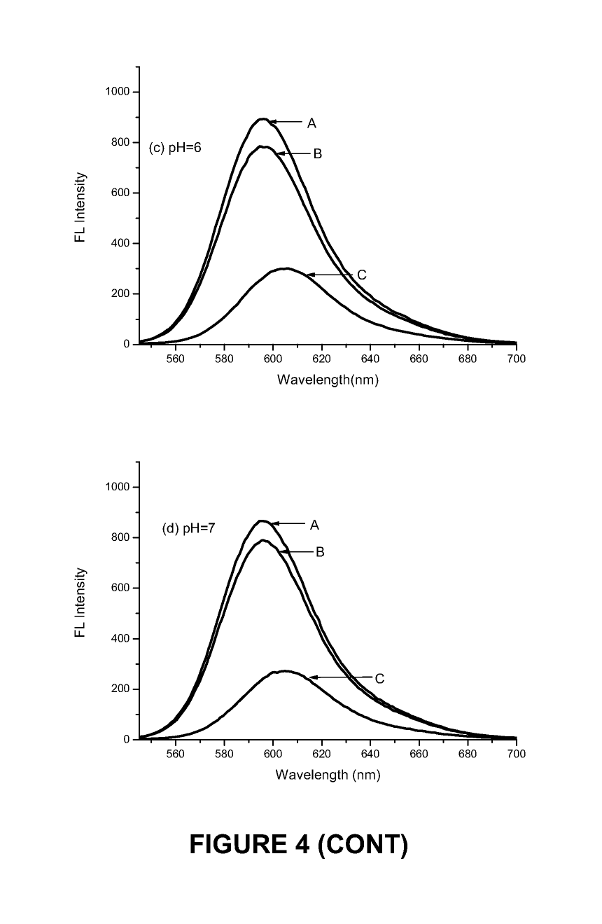

The selectivity of the sensor Compound 1 for Hg²⁺ over environmentally relevant alkali, alkaline earth and transition metal ions (K⁺, Na²⁺, Mg²⁺, Ca²⁺, Ba²⁺, Zn²⁺, Fe²⁺, Ni²⁺, Cd²⁺, Pb²⁺, Cu²⁺) was investigated in different pH solution pH=4, 5, NaOAc 0.02M buffer, pH=6, PBS 0.02M buffer, pH 7 water (panel A pH 4.0, panel B pH 5.0, panel C pH 6.0, panel D pH 7.0). Fluorescence spectra of a 1.0 μM Compound 1 solution in the absence (A line) and presence (B line) of a mixture of all 11 metal ions (each 5 μM). Then addition of 2.0 μM Hg²⁺ to the mixed solution resulted in fluorescence quenching (C line). See FIG. 4.

### Example 6

### Job Plot

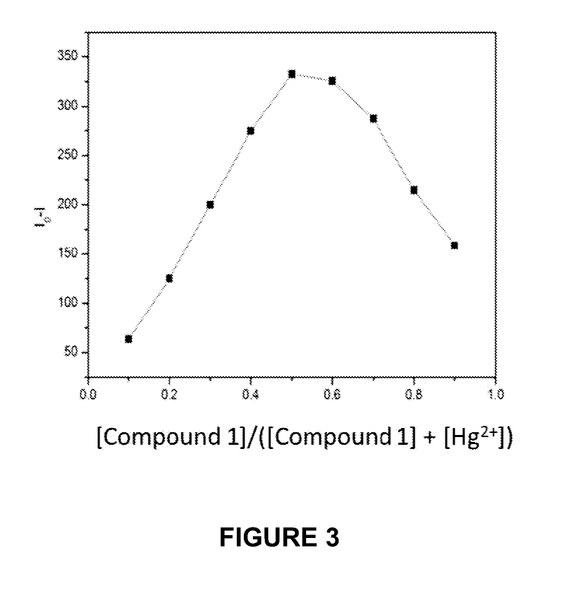

The 1:1 stoichiometry of the complex formed between Hg²⁺ and Compound 1 was determined by a Job plot, which was obtained by measuring the difference in relative fluorescence intensity at 596 nm with the change in molar fraction of Compound 1. Total concentration of Compound 1 and Hg²⁺ was kept constant at 5 μM in aqueous solution. See FIG. 3.

### Example 7

### Solubility of Compound 1

Compound 1 is a dark red color solid with solubility in water about 30 μM, much higher than detection concentration (0.3˜5 μM) that is commonly used in fluorometer or portable detector. Various solvents have been tested in the following table. For transferring Compound 1 as aliquots, ethanol-dichloromethane (DCM) (5:1) is a good solvent giving good solubility and easy evaporation of solvent.

| Solvent | Solubility | Solvent | Solubility |
|---|---|---|---|
| DMF | >200 μM | 15% Glycerol-H₂O | ~50 μM |
| DMF-H₂O (7:3) | >200 μM | 30% Glycerol-H₂O | >86 μM |
| Ethanol-DCM | >342 μM | 50% Glycerol-H₂O | >86 μM |
| (5:1) | | | |
| Ethanol | ~100 μM | 30% Propylene glycol- | >86 μM |
| | | H₂O | |
| isopropanol | >86 μM | 50% Propylene glycol- | >86 μM |
| | | H₂O | |
| Water | ~30 μM | | |

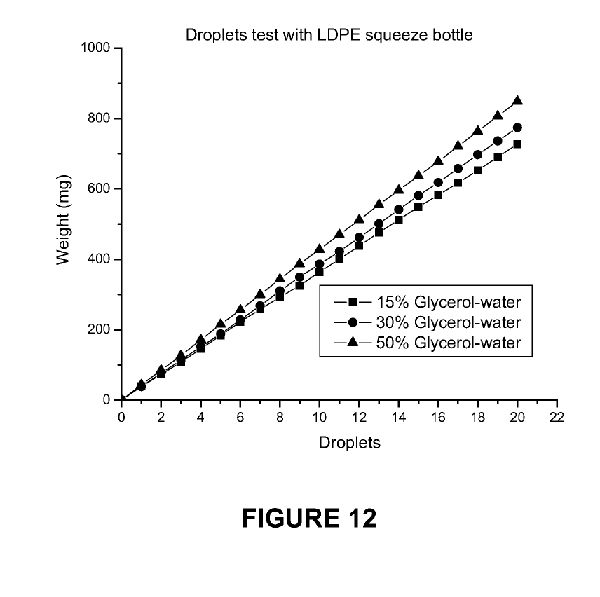

It was also discovered that droplet consistency and uniformity was best with a 50% glycerol-water solution, with variation less than 3%. If five drops of sensor stock solution were used, the variation could be as low as 1-2% (FIG. 12).

### Example 8

### Spectral Properties of Compound 1

The table below shows the maximum absorbance and emission wavelengths as well as the extinction coefficient for Compound 1 in various solvents.

| Solvent | λ_{max,abs} (nm) | ε(M⁻¹cm⁻¹) | λ_{max,flu} (nm) |
|---|---|---|---|
| DMF-H₂O (7:3) | 561 | 1.89 × 10⁴ | 595 |
| Ethanol-DCM (5:1) | 558 | 1.6 × 10⁴ | 595 |
| H₂O | 563 | 1.2375 × 10⁴ | 595 |
| Ethanol | 557 | 1.283 × 10⁴ | 594 |
| Isopropanol | 561 | 1.52 × 10⁴ | 594 |
| 15% Glycerol-H₂O | 564 | 1.5 × 10⁴ | 598 |
| 30% Glycerol-H₂O | 566 | 1.697 × 10⁴ | 598 |
| 50% Glycerol-H₂O | 565 | 1.633 × 10⁴ | 598 |

### Example 9

### Temperature Effect on Fluorescence Quenching of Compound 1

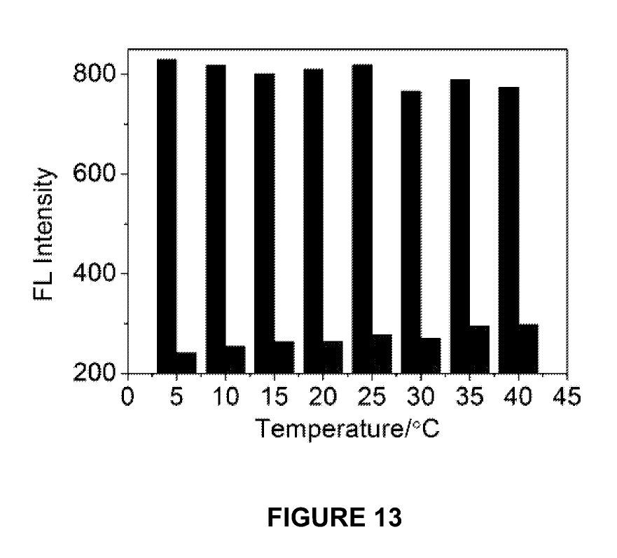

New sensors could be used to test water sources in quite different locations, at different seasons, and from diverse water sources. Compound 1 demonstrated that temperature had a small, but potentially significant, effect on quenching efficiency by one point assay (FIG. 13).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference and as far as they are consistent with the disclosure herein. The mere mentioning of the publications and patent applications does not necessarily constitute an admission that they are prior art to the instant application.

1. A compound comprising a polycyclic aryl group (PAC), wherein at least one solubilizing group and at least one metal binding ligand are covalently bonded to (PAC).

2. The compound of claim 1 wherein the compound comprises formula (I):
(X-L₁)ₙ-(PAC)-(L₂-Z)ₘ (I)
or a salt or solvate, thereof, wherein
L₁ is a linker that covalently bonds each X to (PAC);
L₂ is a linker that covalently bonds each Z to (PAC);
X is a solubilizing moiety;
n is from 1-10;
Z is a metal binding ligand;
m is from 1-10; and
(PAC) is a polycyclic aryl group.

3. The compound of claim 2 wherein (PAC) is perylene or a perylene derivative or analog having 2 or more of the carbons of the fused polycyclic ring system replaced with a heteroatom selected from the group consisting of oxygen, sulfur, and nitrogen.

4. The compound of claim 2 wherein (PAC) comprises a tetracarboxylic diimide.

5. The compound of claim 2 wherein (PAC) is N-(((2R,3S,5R)-3-hydroxy-5-(5-methyl-2,4-dioxo-3,4-dihydropyrimidin-1(2H)-yl)tetrahydrofuran-2-yl)methyl)-3-(9-(3-((((2S,3R,5S)-3-hydroxy-5-(5-methyl-2,4-dioxo-3,4-dihydropyrimidin-1 (2H)-yl)tetrahydrofuran-2-yl)methyl)amino)-3-oxopropyl)-1,3,8,10-tetraoxo-5,6,12,13-tetrakis(pyridin-3-yloxy)-9,10-dihydroanthra[2,1,9-def:6,5,10-d′e′f′]diisoquinolin-2(1H,3H,8H)-yl)propanamide or 2-(((2R,3S,5R)-3-hydroxy-5-(5-methyl-2,4-dioxo-3,4-dihydropyrimidin-1(2H)-yl)tetrahydrofuran-2-yl)methyl)-9-(((2S,3R,5S)-3-hydroxy-5-(5-methyl-2,4-dioxo-3,4-dihydropyrimidin-1(2H)-yl)tetrahydrofuran-2-yl)methyl)-5,6,12,13-tetrakis(pyridin-3-yloxy)anthra[2,1,9-def:6,5,10-d′e′f′]diisoquinoline-1,3,8,10(2H,9H)-tetraone.

6. The compound of claim 2 wherein (PAC) is perylene-3,4,9,10-tetracarboxylic diimide.

7. The compound of claim 1 wherein the solubilizing group is a water solubilizing group.

8. The compound of claim 1 wherein when n is greater than or equal to 2, X is the same water solubilizing moiety.

9. The compound of claim 1 wherein when n is greater than or equal to 2, X is at least two different water solubilizing moieties.

10. The compound of claim 1 wherein X is pyridyl, alkyl pyridinium, substituted pyridyl, substituted alkyl pyridinium, or substituted phenyl.

11. The compound of claim 1 wherein X is a residue of an organic acid or an inorganic acid.

12. The compound of claim 1 wherein X is a polyalkylene oxide.

13. The compound of claim 1 wherein X is a polyalkylene oxide comprising from 1 to 20 ethylene oxide units.

14. The compound of claim 1 wherein each L₁ independently is selected from alkylenyl, —O—, —S—, oligoethylene oxide, or —NR₁₀—, where R₁₀ is hydrogen or an alkyl group.

15. The compound of claim 1 wherein each L₂ is (C1-C12)alkylenyl wherein one or more carbon atoms of the alkylenyl group can be substituted with (1) oxo or halo; (2) one or more carbon atoms can be replaced with an oxygen, sulfur, or nitrogen; or (3) two or three carbon atoms of the alkylenyl group can be taken to together to form a 4-, 5-, or 6-membered heterocyclic or cycloalkyl ring which can be optionally substituted, or a combination thereof.

16. The compound of claim 1 wherein each L₂ comprises one or more amino acid moieties or a derivative thereof.

17. The compound of claim 1 wherein L₂ has the formula 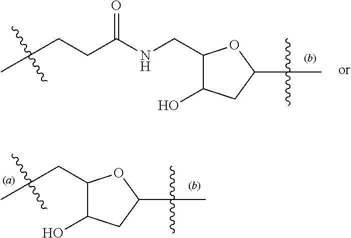 [SMILES: *CCC(=O)NCC1OC(*)CC1O ; *CC1OC(*)CC1O]
wherein bond (a) is bonded to (PAC) and bond (b) is bonded to Z.

18. The compound of claim 1 wherein each Z independently is 1H-pyrimidine-2,4-dione or a derivative or analog thereof.

19. The compound of claim 1 wherein each Z independently has the formula 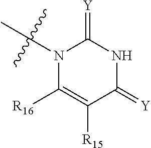
wherein each R₁₅ and R₁₆ is, independently, hydrogen, a halide, an alkyl group, or a haloalkyl group; and
each Y is, independently, oxygen or sulfur.

20. The compound of claim 19 wherein each Y is oxygen, R₁₆ is hydrogen, and R₁₅ is methyl for each Z.

21. The compound of claim 1 wherein the compound has the formula (II): 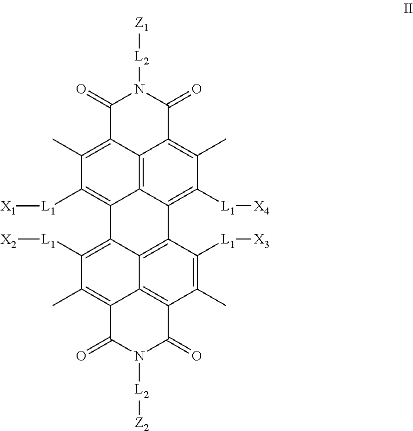
or a salt, solvate, or clathrate thereof, wherein
each L₁ independently is a linker that covalently connects the solubilizing moieties X₁, X₂, X₃, and X₄ to the polycyclic aryl group;
each L₂ independently is a linker that covalently connects a metal ion binding moiety Z₁ and Z₂, to the polycyclic aryl group;
each of X₁, X₂, X₃, and X₄ independently is a solubilizing moiety that is an optionally substituted heterocyclyl group or a substituted aryl group; and
each of Z₁ and Z₂ independently is a metal ion binding moiety that is 1H-pyrimidine-2,4-dione or a derivative or analog thereof.

22. The compound of claim 21, wherein X₁, X₂, X₃, and X₄ are the same group.

23. The compound of claim 22, wherein X₁, X₂, X₃, and X₄ are each a substituted or unsubstituted pyridinium group, wherein the pyridinium group is neutral or the salt thereof.

24. The compound of claim 22, wherein X₁, X₂, X₃, and X₄ are each a phenyl group substituted with at least one hydrophilic group.

25. The compound of claim 24, wherein the hydrophilic group comprises a sulfate group, a phosphonate group, or an oligoethylene oxide.

26. The compound of claim 21, wherein each L₁ is alkylenyl, —O—, —NH—, —S—, and oligoethylene oxide.

27. The compound of claim 21 wherein each L₂ has the formula 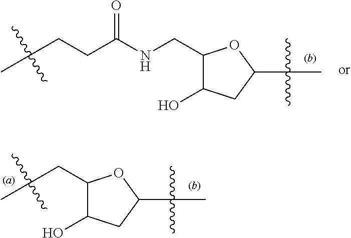 [SMILES: *CCC(=O)NCC1OC(*)CC1O ; *CC1OC(*)CC1O]
wherein bond (a) is bonded to (PAC) and bond (b) is bonded to Z₁ and Z₂.

28. The compound of claim 21 wherein each Z₁ and Z₂ has the formula 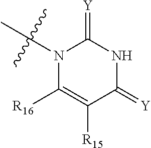
wherein each R₁₅ and R₁₆ is, independently, hydrogen, a halide, an alkyl group, or a haloalkyl group; and
each Y is, independently, oxygen or sulfur.

29. The compound of claim 28 wherein each Y is oxygen, R₁₆ is hydrogen, and R₁₅ is methyl for each Z.

30. The compound of claim 1, wherein the compound is Compound 1, Compound 2, Compound 3, Compound 4, Compound 5, or Compound 6.

31. A complex comprising a compound of claim 1 and a metal ion bound to the metal binding ligand.

32. The complex of claim 31, wherein the metal ion comprises Cu²⁺, Ni²⁺, Fe²⁺, Fe³⁺, Pb²⁺, Cd²⁺, Hg²⁺, Zn²⁺, Mn²⁺, Ba²⁺, Mg²⁺, Ca²⁺, Cr²⁺, Co²⁺, an ion of arsenic (As⁵⁺, As³⁺), antimony (Sb⁵⁺ or Sb³⁺), or thallium (Tl⁺ or Tl³⁺), a rare earth metal ion, a lanthanide metal ion, an actinide metal ion, or any combination thereof.

33. The complex of claim 31, wherein the metal ion is Hg²⁺.

34. The complex of claim 31, wherein the complex is a clathrate.

35. The complex of claim 31, wherein the complex has lower fluorescence compared to the metal sensor compound in the absence of the metal ion.

36. A composition comprising the compound of claim 1 and a solvent.

37. The composition of claim 36 wherein the solvent comprises water, an organic solvent, or a mixture thereof.

38. The composition of claim 36 wherein the solvent comprises a mixture of water and an organic solvent.

39. The composition of claim 38 wherein the organic solvent comprises an alcohol, a glycol, dimethyl formamide, dichloromethane, dimethylsulfoxide, dimethylacetamide, acetone, tetrahydrofuran, dioxane, or any combination thereof.

40. The composition of claim 38 wherein the organic solvent is glycerol or propylene glycol.

41. A composition comprising the compound of claim 1 and a water miscible, non-volatile organic polymer.

42. The composition of claim 41, wherein the water miscible, non-volatile organic polymer comprises a polyalkylene alcohol, a poloxamer, polyvinyl alcohol, polyvinylpyrrolidone, poly (N-vinyl lactam), a polyacrylamide, a polyanhydride, a polyacrylic acid, a polyvinyl ether, polyethyleneimine, cellulose or a derivative thereof, or any combination thereof.

43. A method of detecting a metal ion in a test sample, the method comprising
contacting the test sample with the compound of claim 1 and measuring the fluorescence of the test sample;
comparing the fluorescence of the test sample to the fluorescence of a control sample containing only the compound of claim 1,
wherein the difference in fluorescence between the test sample and the control sample indicates the presence of the metal ion in the test sample.

44. A method for quantifying a metal ion concentration in a test sample, the method comprising
contacting the test sample with a calibration solution comprising a known concentration of compound of claim 1 and measuring the fluorescence of the test sample;
calibrating the fluorescence of the test sample to a calibration curve to determine the concentration of the metal ion in the test sample.

45. The method of claim 43 further comprising the step exciting or irradiating the compound at a wavelength in the range of 180 nm to 700 nm prior to contacting the test sample with the compound.

46. The method of claim 43 wherein said test solution comprises one or more different metal ions selected from Cu²⁺, Ni²⁺, Fe²⁺, Fe³⁺, Pb²⁺, Cd²⁺, Hg²⁺, Zn²⁺, Mn²⁺, Ba²⁺, Mg²⁺, Ca²⁺, Cr²⁺, Co²⁺, an ion of arsenic (As⁵⁺, As³⁺), antimony (Sb⁵⁺ or Sb³⁺), or thallium (Tl⁺or Tl³⁺), a rare earth metal ion, a lanthanide metal ion, an actinide metal ion, or any combination thereof.

47. The method of claim 43 wherein the test sample is a liquid stream or a gas stream.

48. The method of claim 43 wherein the test sample comprises water, an aqueous based solution, air, hydrogen, natural gas, exhaust gas from the thermal destruction of chemical warfare munitions, liquid hydrocarbons, wastewater discharge chlor-alkali plant, waste streams from dye, pharmaceutical, and agrochemical manufacturing, waste streams from mining, waste streams from concrete or cement production, waste streams from production of printed circuit boards or other electroplating processes, waste generated during thermometer or vacuum pump gauge manufacturing, exhaust gas passed through a water scrubber, an air stream produced from an air purification system, waste materials generated from the production of nuclear weapons, or an offgas from (1) a mixed waste incineration, (2) a plasma enhanced melter, and (3) ventilation of a hot cell.

49. The method of claim 43 wherein the test sample comprises municipal water supplies, personal & domestic water supplies, river water, lake water, pond water, groundwater, or a soil extract.
